(19) United States
(12) Patent Application Publication
(10) Pub. No.: US 2016/0206192 A1
(43) Pub. Date: Jul. 21, 2016

(54) ARITHMETIC PROCESSOR AND CONTROL METHOD THEREOF

(71) Applicant: CANON KABUSHIKI KAISHA, Tokyo (JP)

(72) Inventor: Eito Sakakima, Kamakura-shi (JP)

(21) Appl. No.: 14/994,790

(22) Filed: Jan. 13, 2016

(30) Foreign Application Priority Data
Jan. 16, 2015 (JP) 2015-006963
Jan. 21, 2015 (JP) 2015-009587

(51) Int. Cl. A61B 3/06; A61B 3/00

(52) U.S. Cl. CPC A61B 3/066; A61B 3/0025

(57) ABSTRACT

An arithmetic processor including: a selection unit configured for a user to select a color that satisfies a predetermined condition with respect to a reference color formed on a medium that reflects light, out of a plurality of different colors displayed on a display apparatus; a determination unit configured to determine colors to be displayed on the display apparatus next time, based on the color selected by the user; and a calculation unit configured to calculate a visual characteristic of the user, based on results of repeating the determination of colors by the determination unit and the selection by the user for a plurality of times. In a case where the user repeatedly selects a color out of a combination of colors, the calculation unit calculates the visual characteristic of the user, based on the colors included in the combination.

### BACKGROUND OF THE INVENTION

1. Field of the Invention

The present invention relates to an arithmetic processor and a control method thereof.

2. Description of the Related Art

In recent years, in a color management display apparatus having high color gamut and high resolution, a soft proofing function for reproducing the colors of a printed matter and for checking the colors has been considered. By using the soft proofing function, an image can be edited while checking the actual print image on the display apparatus, and the finished work can be checked. Therefore operation efficiency can be improved, including the reduction of operation time.

On the other hand, it is known that a visual characteristic is different depending on the individual. Here the visual characteristic is referred to as a “matching color function”, and is expressed as a curve to indicate sensitivity to light depending on each wavelength. The color an individual perceives is determined by the color matching function and the spectral radiance of the observation target. Therefore if the visual characteristic represented by the color matching function is different, the color to be perceived is also different. Furthermore, the difference of the color to be perceived changes considerably depending on the waveform of the spectral radiance of the target. If the spectral radiance changes dramatically within a specific narrow range of a wavelength region, in other words if the wavelength acutely changes, the change of perceptible quantity, due to the difference represented by the color matching function, tends to increase.

To increase the color gamut of the expressible colors, liquid crystal display apparatuses that use an LED (Light Emitting Diode) as the backlight are becoming popular. However in the case of an LED, the profile of the spectral radiance is steep, hence liquid crystal display apparatuses using an LED backlight could be devices of which perceptible quantity tends to be different, depending on the personal difference of the visual characteristic. In the case of a high color rendering fluorescent light, which has been used as a light source to observe printed matter, on the other hand, has a waveform of spectral radiance which gently changes. Therefore if a high color rendering fluorescent light is used as a light source to observe printed matter, the perceptible quantity is not very different depending on the user.

If colors generated by the display apparatus are perceived differently depending on the individual, a major problem occurs in an operation to accurately evaluate colors by soft proofing or the like. For example, in the case when colors of the work to be published eventually as printed matter are determined by observing the colors on the display apparatus, if the perceived colors are different depending on the user, each user may determine and process colors based on the colors which are perceived differently depending on the user. Therefore when the final printed matter is created, colors which are different from the intended colors may be perceived, and the quality of the work is diminished.

To solve this problem, a technique disclosed in Japanese Patent Application Laid-Open No. 2005-109583 proposes that when an image is displayed on a display apparatus, display of the display apparatus is corrected using a pre-measured visual characteristic, whereby the personal difference of the perceivable quantity is reduced.

Further, many color management display apparatuses include a function to adjust the display image quality, hence the user themselves can make adjustments so that the colors on paper and the colors on the display apparatus match.

### SUMMARY OF THE INVENTION

According to the method disclosed in Japanese Patent Application Laid-Open No. 2005-109583 and in the general methods for correcting a visual characteristic utilizing the image quality adjustment function of display apparatuses, correction of the display apparatus is performed to the optimum for the user by presenting reference colors to the user, and acquiring information on how the reference colors are perceived by the user. A following method, for example, is under consideration.

A plurality of color chips having a predetermined color difference are simultaneously displayed on the display apparatus, and the user compares the color chips with the printed matter to be a reference, and selects the color chips of which colors match with the colors of the printed matter. If the printed matter is observed under a high color rendering fluorescent light source, fluctuation of perceptible quantity of the user can be decreased, whereby the color chips can be used as reference colors. By repeating this selection processing while gradually decreasing the color difference between the color chips displayed on the display apparatus, colors on the display apparatus that match with the printed matter for the user can be specified. The colors on the display apparatus that match with those of the printed matter determine the differences from the reference values for this user, and become the values that can be handled as the visual characteristic of this user. By correcting the colors of the display apparatus using the acquired visual characteristic, display of the display apparatus can be corrected to the optimum for the user.

A method for measuring the visual characteristic will be described with reference to the drawings. FIG. 1 is a diagram depicting a method for measuring a personal visual characteristic.

101 in FIG. 1 denotes a high color rendering fluorescent light to irradiate a comparison reflector 103 to measure the visual characteristic. If the comparison reflector 103, on which reference color chips 104 are printed, is irradiated by the high color rendering fluorescent light 101, colors, of which difference of perceptible quantity depending on the user is small, are reflected. Therefore the reference color chips 104 can be used as reference to acquire the visual characteristic. The visual characteristic of the user is measured by displaying a plurality of measurement color chips 105 having a predetermined color difference on the display apparatus 102, and having the user select a color close to the reference color chip 104. By gradually decreasing the color difference between the respective colors of the measurement color chips 105 to be displayed on the display apparatus 102, the visual characteristic of the user can be acquired with a high degree of accuracy.

In the above mentioned measurement of the visual characteristic, some users may be unable to select a color matching with the colors of the measurement color chips 105 displayed on the display apparatus. This will be described with reference to a drawing.

FIG. 15 shows diagrams depicting colors of the measurement color chips 105 which are displayed when the visual characteristic is measured. 1500 in FIG. 15 is a diagram expressing the a* axis and the b* axis on a plane in a space indicating colors quantitatively, which is called the L*a*b* space. The L*a*b* space is a color space represented by an index L to indicate brightness and by indexes a* and b* to indicate colors, where colors can be expressed by values on the a* axis and the b* axis. The distance between two points in the L*a*b* space is the color difference, which is digitized as ΔE.

In 1500 in FIGS. 15, 1501 to 1505 indicate points representing a plurality of colors, which are colors corresponding to the measurement color chips 105 indicated by 1506 to 1510. When a visual characteristic is measured, a plurality of different colors are presented to the user as the measurement color chips 105, as shown in 1501 to 1505, and the user selects a color chip of which color is close to that of a reference color chip.

Here it is assumed that a match point with the reference color chip 104, which indicates the visual characteristic of the user, is the point indicated by 1511 in FIG. 15. In this case, there are no match points in 1501 to 1505, and the user more likely selected 1503 or 1504 as the match point. If 1501 to 1505 in FIG. 15 are final measurement color chips 105, then a color that is different from the color position at 1511, which should be acquired, is acquired as the visual characteristic. As a result, the visual characteristic value of the user, beyond the color difference between the measurement color chips 105, cannot be acquired, and a highly precise visual characteristic value cannot be acquired.

Further, in the above mentioned measurement of the visual characteristic, a user may perceive a color difference of the measurement color chips displayed on the display apparatus as smaller than an actual difference. In this case, the user may have difficulty in selecting a measurement color chip that matches with the reference color chip, resulting in a lengthy measurement time. Therefore with the technique disclosed in Japanese Patent Application Laid-Open No. 2005-109583, in some cases a personal visual characteristic cannot be efficiently measured.

With the foregoing in view, it is an object of the present invention to provide a technique to measure the visual characteristic of a user at high precision.

It is another object of the present invention to shorten the time required for measuring the visual characteristic of the user.

A first aspect of the present invention is an arithmetic processor, including: a selection unit configured for a user to select a color that satisfies a predetermined condition with respect to a reference color formed on a medium that reflects light, out of a plurality of different colors displayed on a display apparatus; a determination unit configured to determine colors to be displayed on the display apparatus next time, based on the color selected by the user; and a calculation unit configured to calculate a visual characteristic of the user, based on results of repeating the determination of colors by the determination unit and the selection by the user for a plurality of times. When the user repeatedly selects a color out of a combination of two or more colors for a fixed number of times or more, the calculation unit calculates the visual characteristic of the user, based on the colors included in the combination.

A second aspect of the present invention is a control method for an arithmetic processor, including: a selection step in which a user selects a color that satisfies a predetermined condition with respect to a reference color formed on a medium that reflects light, out of a plurality of different colors displayed on a display apparatus; a determination step in which colors to be displayed on the display apparatus next time are determined, based on the color selected by the user; and a calculation step in which a visual characteristic of the user is calculated, based on the results of repeating the determination of colors in the determination step and the selection of colors by the user for a plurality of times. When the user repeatedly selects a color out of a combination of two or more colors for a fixed number of times or more, the visual characteristic of the user is calculated, based on the colors included in the combination in the calculation step.

A third aspect of the present invention is an arithmetic processor including: a selection unit configured for a user to select a color chip that satisfies a predetermined condition with respect to a reference color out of a plurality of different color chips displayed on a display apparatus; a determination unit configured to determine the color chip selected by the user and one or a plurality of color chips having a color difference from the color chip, as color chip(s) to be displayed on the display apparatus next time; and a calculation unit configured to calculate a visual characteristics of the user, based on history information, which is information on results of repeating the determination of the color chips by the determination unit and the selection of the color chip by the user for a plurality of times. When determination is made that the color chips selected by the user are not converged to one, based on the history information, the determination unit changes the color chips to be displayed on the display apparatus.

A fourth aspect of the present invention is a control method for an arithmetic processor configured to acquire a visual characteristic of a user, including: a selection step in which a user selects a color chip that satisfies a predetermined condition with respect to a reference color, out of a plurality of different color chips displayed on a display apparatus; a determination step in which a color chip selected by the user and one or a plurality of color chips having a color difference from the color chip are determined as the color chips to be displayed on the display apparatus next time; and a calculation step in which the visual characteristic of the user is calculated, based on history information, which is information on results of repeating the determination of the color chips in the determination step and the selection of the color chip by the user for a plurality of times. When determination is made that color chips selected by the user are not converged to one based on the history information, the color chips to be displayed on the display apparatus are changed in the determination step.

According to the present invention, a technique to acquire a visual characteristic of the user at high precision can be provided.

Moreover, according to the present invention, the time required for measuring the visual characteristic of the user can be shortened.

Further features of the present invention will become apparent from the following description of exemplary embodiments with reference to the attached drawings.

### BRIEF DESCRIPTION OF THE DRAWINGS

FIG. 1 is a diagram depicting composing elements of an example;

FIG. 2 is a diagram depicting functional blocks of Example 1;

FIG. 3 is a flow chart depicting visual characteristic acquisition processing;

FIG. 4A to FIG. 4F are diagrams depicting a method for visual characteristic acquisition processing;

FIG. 5 shows diagrams depicting comparison reflectors which are used upon acquiring a visual characteristic;

FIG. 6A and FIG. 6B are tables for explaining a personal visual characteristic;

FIG. 7 is a flow chart depicting a processing flow of Example 1;

FIG. 8 is a table for explaining selection history information;

FIG. 9A and FIG. 9B are diagrams depicting conversion processing for visual characteristic values;

FIG. 10A and FIG. 10B are diagrams depicting conversion processing for visual characteristic values;

FIG. 11A and FIG. 11B are diagrams depicting conversion processing for visual characteristic values;

FIG. 12 is a diagram depicting functional blocks of Example 2;

FIG. 13 is a flow chart depicting a processing flow of Example 2;

FIG. 14 is a diagram depicting the target precision and calculated precision according to Example 2;

FIG. 15A and FIG. 15B are diagrams depicting a problem;

FIG. 16 is a diagram depicting functional blocks of a display apparatus in Example 3;

FIG. 17 is a flow chart depicting a processing flow of Example 3 and Example 4;

FIG. 18A to FIG. 18D are diagrams depicting a method for detecting a state where the user is having difficulty in selecting a color chip in Example 3;

FIG. 19A and FIG. 19B are diagrams depicting a method for detecting a state where the user is having difficulty in selecting a color chip in Example 3;

FIG. 20A to FIG. 20C are diagrams depicting a method for changing display of the measurement color chips according to Example 3;

FIG. 21A to FIG. 21C are diagrams depicting a method for changing display of the measurement color chips according to Example 3;

FIG. 22A to FIG. 22C are diagrams depicting a method for changing display of the measurement color chips according to Example 3;

FIG. 23A and FIG. 23B are diagrams depicting a method for changing display of the measurement color chips according to Example 4;

FIG. 24A and FIG. 24B are diagrams depicting a display apparatus of Example 5;

FIG. 25A and FIG. 25B are diagrams depicting a method for changing display of the measurement color chips according to Example 5; and

FIG. 26 is a flow chart depicting a processing flow of Example 5.

### DESCRIPTION OF THE EMBODIMENTS

### Example 1

Example 1 is an example of an arithmetic processor that performs processing to acquire a visual characteristic of a user. In the following example, a user compares the reference color chips of a printed matter and measurement color chips of a display apparatus, and repeatedly selects a measurement color chip of which color is close to that of the reference color chip, so as to acquire the visual characteristic of the user, and at this time, the measurement color chip selected by the user is changed using the history of the selection processing.

Example 1 will now be described with reference to the drawings.

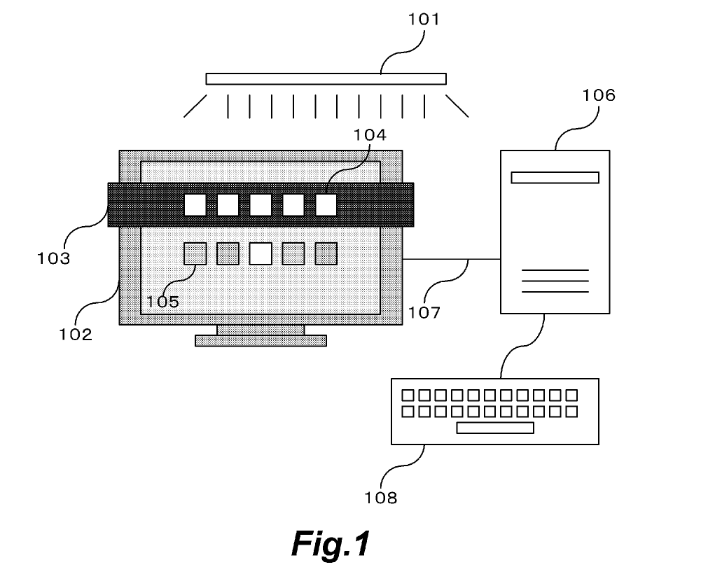

FIG. 1 is a diagram depicting composing elements of Example 1.

101 in FIG. 1 denotes a high color rendering fluorescent light used for observing a printed matter. A color rendering property is defined as an index to represent the capability of a light source to evaluate a color on a reflector. As the color rendering property of the light source becomes higher, the light source includes a spectrum with more wavelength regions, and more accurately reflected light from a printed matter or the like can be obtained. In Example 1, colors printed on a printed matter (e.g. paper medium) are shown as an example of the reference colors formed on a medium that reflects light, but a comparison reflector is not limited to this.

102 in FIG. 1 denotes a display apparatus to display images. A mainstream display apparatus in recent years is a liquid crystal display apparatus, of which backlight is such light sources as an LED and a CCFL (Cold Cathode Fluorescent Lamp).

103 in FIG. 1 denotes a comparison reflector used for acquiring a personal visual characteristic. Reference color chips 104 are printed on the comparison reflector 103, and are irradiated by the high color rendering fluorescent light 101, whereby reference values to acquire the visual characteristic can be generated.

105 in FIG. 1 denotes measurement color chips to compare with the reference color chips 104, in order to acquire a personal visual characteristic. The measurement color chips 105, having a plurality of different colors, are displayed on the display apparatus 102, and the user compares these colors with the reference color chips 104 and selects a color which is perceived as the same, whereby the visual characteristic of the user is acquired. In the example in FIG. 1, five color chips are indicated for both the reference color chips 104 and the measurement color chips 105, but the number of color chips of the present invention is not limited to five.

106 in FIG. 1 denotes a computer for displaying an image on the display apparatus 102. Normally a PC (Personal Computer) is used as the computer 106. The computer 106 also controls the visual characteristic measurement.

107 in FIG. 1 denotes an image signal line for inputting image signals from the computer 106 to the display apparatus 102. For the image signal line 107, normally a DP (Display Port) cable or the like is used.

108 in FIG. 1 denotes a user interface, which is connected to the computer 106, and receives input from the user. Examples of the user interface 108 are a mouse and a keyboard, and selection information of the measurement color chips 105 or the like is inputted through these user interfaces 108.

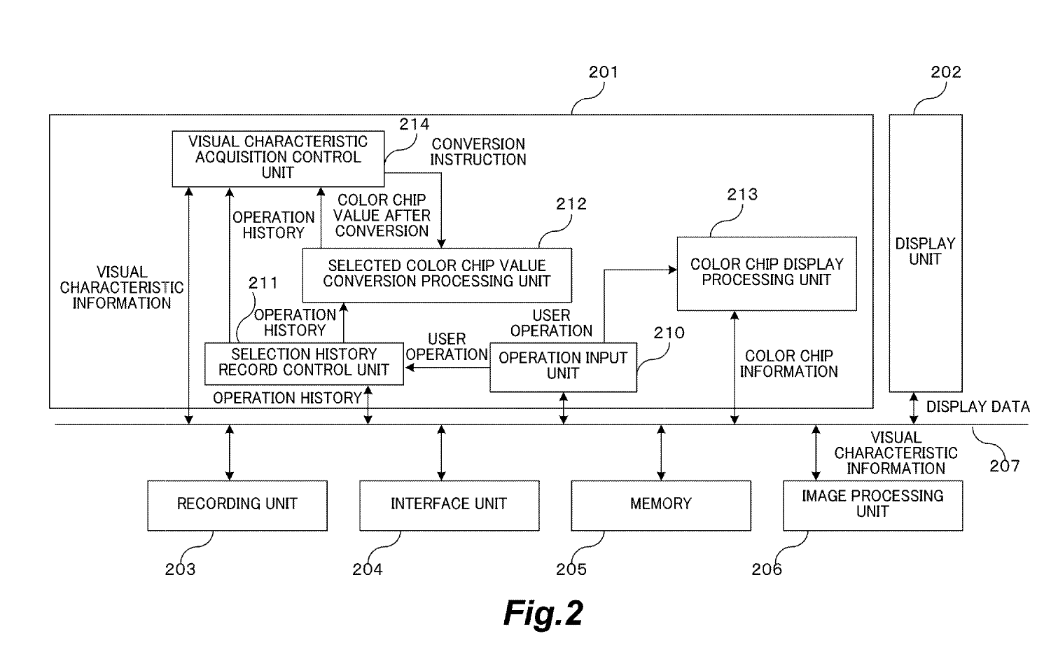

FIG. 2 is a diagram depicting functional blocks of Example 1. Each functional block will now be described.

201 in FIG. 2 denotes a CPU (Central Processing Unit) which performs various controls. The CPU 201 performs control upon acquiring a visual characteristic, later-mentioned selected color chip value conversion processing and the like. The CPU 201 also executes other controls, such as reading various data.

202 in FIG. 2 denotes a display unit for displaying images, and corresponds to the display apparatus 102 in FIG. 1.

203 in FIG. 2 denotes a recording unit that saves the image information to be displayed on the display unit 202, and the acquired visual characteristics. The recording unit 203 also records various control programs.

204 in FIG. 2 denotes an interface unit that receives input from the user. The interface unit has a function to receive information inputted via the user interface 108 in FIG. 1, and to transfer information to the CPU 201 or the like.

205 in FIG. 2 denotes a memory which temporarily stores intermediate data of various operation results and the like.

206 in FIG. 2 denotes an image processing unit that performs various processing operations to image signals to be displayed on the display unit 202. In the image processing unit 206, conversion processing to appropriate colors for the user is executed using the acquired visual characteristic values.

207 in FIG. 2 denotes a bus which is used for transmitting/receiving various data.

An operation input unit 210 in FIG. 2 acquires information on the operation performed by the user, which is transferred from the interface unit 204. The information on the operation performed by the user is transferred to the later mentioned selection history record control unit 211, and is recorded as selection history information.

When a visual characteristic is acquired, the selection history record control unit 211 in FIG. 2 performs a control to save information on the measurement color chip 105 selected by the user as history information. The selection history information is recorded in the recording unit 203. The selection history information will be described in detail later.

A selected color chip value conversion processing unit 212 in FIG. 2 performs processing to convert the value of the measurement color chip 105 selected by the user into a value closer to the visual characteristic of the user using the selection history information.

A color chip display processing unit 213 in FIG. 2 performs processing to actually display the measurement color chips 105 on the display apparatus 102. In concrete terms, the color chip display processing unit 213 acquires from the operation input unit 210 information on the measurement color chip 105 selected by the user, determines the colors of the measurement color chips 105 to be displayed next, and writes this color chip information to a frame buffer created in the memory 205.

A visual characteristic acquisition control unit 214 in FIG. 2 performs a control to acquire visual characteristic values of the user using the selection history information of the measurement color chip 105 recorded in the selection history record control unit 211.

Now a visual characteristic measurement method of Example 1 will be described with reference to the drawings.

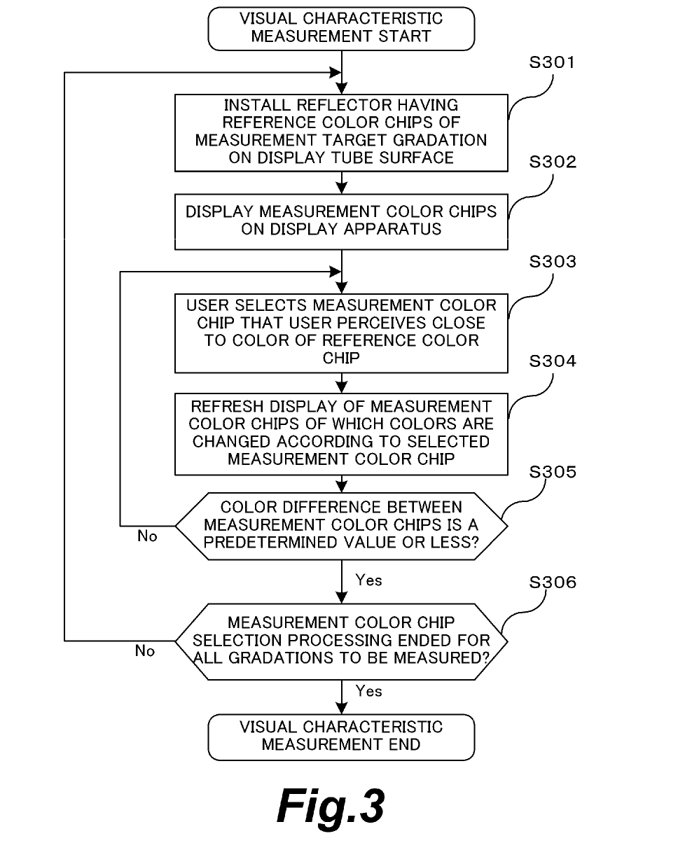

FIG. 3 is a flow chart depicting the processing flow to be a basis for the visual characteristic acquisition method of Example 1. The processing flow in FIG. 3 is controlled by the visual characteristic acquisition control unit 214 in FIG. 2.

In step S301, the user installs the comparison reflector 103 in FIG. 1 to the tube surface of the display apparatus 102.

In step S302, the color chip display processing unit 213 displays the measurement color chips 105 for the user to compare on the display apparatus 102. Here the positional relationship between the reference color chips 104 on the comparison reflector 103 and the measurement color chips 105 are as shown in FIG. 1, for example, so that the user can easily compare the reference color chips 104 and the measurement color chips 105 on the display.

In step S303, the user performs processing of selecting a measurement color chip 105 of which color is perceived closest to that of the reference color chip 104.

In step S304, the color chip display processing unit 213 determines the colors to be displayed on the display apparatus 102 next time, based on the color of the measurement color chip 105 selected by the user. In Example 1, the color chip display processing unit 213 adjusts the colors to be displayed on the display apparatus 102 next time based on the color of the measurement color chip 105 selected by the user, and refreshes the display of the measurement color chips 105 of which colors are changed according to adjustment. The colors of the measurement color chips 105 are determined by the color chip display processing unit 213 according to the measurement color chip 105 selected by the user.

In step S305, the visual characteristic acquisition control unit 214 determines whether the color difference between the plurality of measurement color chips 105 is a threshold or less, and if a threshold or less, the processing advances to step S306. If not a threshold or less, the processing in step S303 and the processing in step S304 are repeated.

In step S306, the visual characteristic acquisition control unit 214 determines whether the selection processing by the user ended for all the reference color chips 104. If the selection processing ended for all the reference color chips 104, the visual characteristic acquisition control unit 214 ends the visual characteristic measurement. Therefore in Example 1, the visual characteristic of the user is calculated based on: the result of repeating the selection of the color chip by the user; and the determination of the colors of the color chips to be displayed next based on the color of the color chip selected by the user.

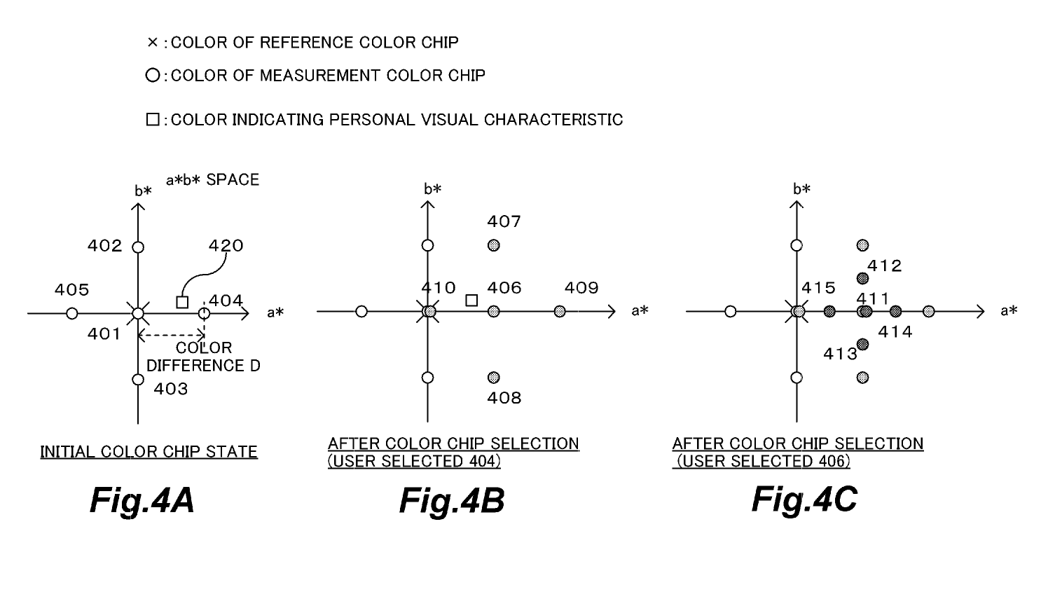

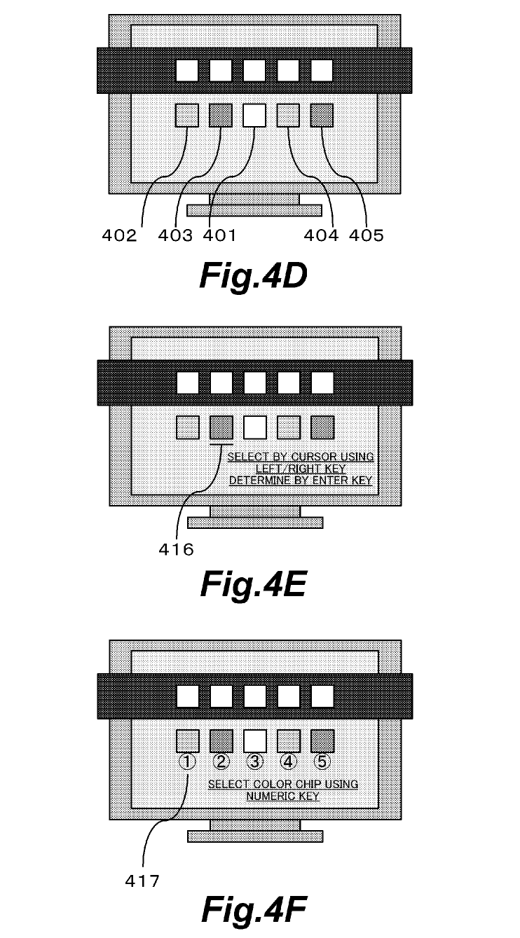

The processing operations executed in step S303 and in step S304 will be described in detail with reference to FIG. 4. FIG. 4 are diagrams depicting how the colors of a plurality of measurement color chips 105 change by the selection processing performed by the user in step S303.

FIG. 4A is a diagram depicting the colors of the measurement color chips 105 which are initially displayed. The two dimensional space shown in FIG. 4A to FIG. 4C is an L*a*b* space having two axes, a* and b*, expressed on a plane. The L*a*b* space is a color space represented by an index L to indicate brightness and by indexes a* and b* to indicate colors, where colors can be expressed as values on the a* axis and the b* axis. The distance between two points in the L*a*b* space is the color difference, which is digitized as ΔE.

In FIG. 4A, the position indicated by X indicates the color of the reference color chip 104, and the positions indicated by ◯ indicate the colors of the measurement color chips 105. FIG. 4 shows an example when a number of measurement color chips 105 is five. In the initial arrangement, the measurement color chips 105 are set at four points which are distant from the color of the reference color chip 104 by a predetermined color difference D in the a* axis and the b* axis, and at a position which indicates the same color as the reference color chip 104. In concrete terms, 401 has the same color as the reference color chip 104, and 402 to 405 are colors which are distant from the reference color chip 104 by a predetermined color distance D. “Same color” here refers to colors having the same values when measured using a measuring instrument.

Here it is assumed that the position indicating the visual characteristic of the user to be measured is the color indicated by □. By acquiring the color of 420 in FIG. 4 indicated by □, it is known how the color 401 of the reference color chip 104 is perceived by this user on the display apparatus 102, whereby the visual characteristic of the user can be acquired.

In the case of the user performing processing to select a color closest to the reference color chip 104, if 420 in FIG. 4 is the position of the color indicating the visual characteristic of the user, the measurement color chip 105 to be selected is 404 in FIG. 4A. And if the user selects 404, the arrangement of the measurement color chips 105 is changed, as shown in FIG. 4B. In FIG. 4B, 406 to 410 are positions indicating the colors of the measurement color chips 105 to be displayed after the display is refreshed. The colors of the measurement color chips 105 displayed here are colors having a predetermined color difference from colors around a position of the color selected by the user. The position indicating the same color as 404 is 406. In this state, the user performs the selection processing again. In the state of FIG. 4B, the user probably selects 406, which is close to the position of 420. If 406 is selected, this means that a color the same as 404, which was selected in the state of FIG. 4A, is repeatedly selected, and it is presumed that the visual characteristic of this user is located around 406. Therefore the colors of the measurement color chips 105 to be displayed next time becomes 412 to 415, of which color difference from 406 is smaller than last time, as shown in FIG. 4C. The color of 411 is the same as the color of 406. By repeating the selection processing described in FIG. 4A to FIG. 4C, the display colors of the measurement color chips 105 approach 420, which is a color indicating the visual characteristic of the user, while gradually decreasing the color difference among the measurement color chips 105. Then the selection processing by the user is repeated until the color difference among the measurement color chips 105 becomes a threshold or less, whereby the visual characteristic is specified.

For the user to select a measurement color chip 105, a cursor to select a measurement color chip 105 may be displayed, for example, as indicated by 416 in FIG. 4E. A number may be assigned to each one of the measurement color chips 105, as indicated by 417 in FIG. 4F, so that the number can be selected by input using a numeric keypad or the like. In this way, the processing to select the measurement color chip 105 in S303 and S304 in FIG. 3 is performed by the method indicated in FIG. 4.

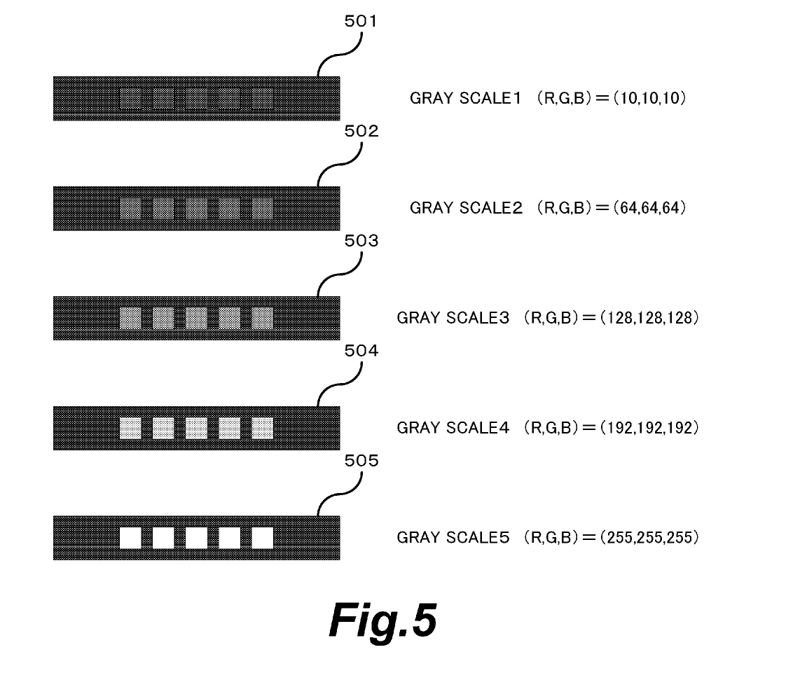

An example of the reference color chips 104 will be described with reference to FIG. 5. FIG. 5 shows diagrams depicting examples of five reference color chips 104 having five types of grayscales. A grayscale is selected as a reference color chip 104 because the difference between personal visual characteristic is especially conspicuous in achromatic colors, and it is effective to acquire a visual characteristic based on grayscales. In the example in FIG. 5, the visual characteristic is acquired using five gradations, but the present invention is not limited to this. If five gradations are used, the processing that started from step S301 is repeated five times in the flow chart of FIG. 3.

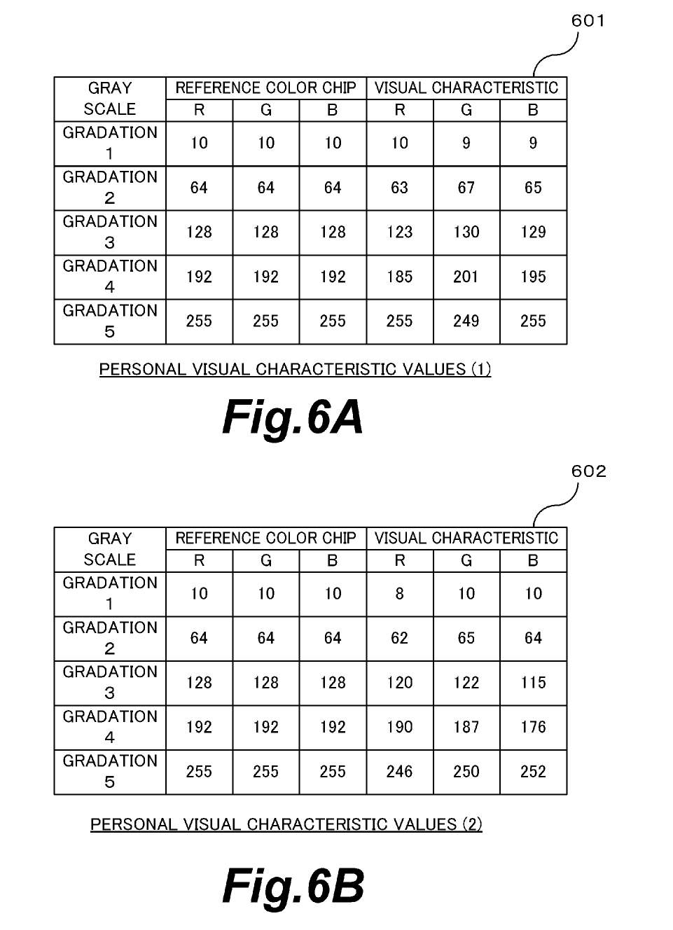

FIG. 6 shows examples of visual characteristic values acquired by the visual characteristic acquisition flow shown in FIG. 3. FIG. 6 are tables to show personal visual characteristic values corresponding to the RGB values which indicate the respective reference color chip values in a plurality of grayscales. 601 and 602 in FIG. 6 show the visual characteristics of different users. Even if the RGB values of the reference color chip values are the same, different RGB values are selected depending on the user. In the image processing unit 206 in FIG. 2, colors are converted using the visual characteristic values in FIG. 6, and the color display suitable for the user is implemented. There are various color conversion methods based on visual characteristics, such as changing the colors to be displayed on the display apparatus 102 according to the table in FIG. 6 on gray colors. In the case of changing only gray colors, the gradation values that do not exist in the table in FIG. 6 are converted into values determined by interpolation processing, such as linear interpolation.

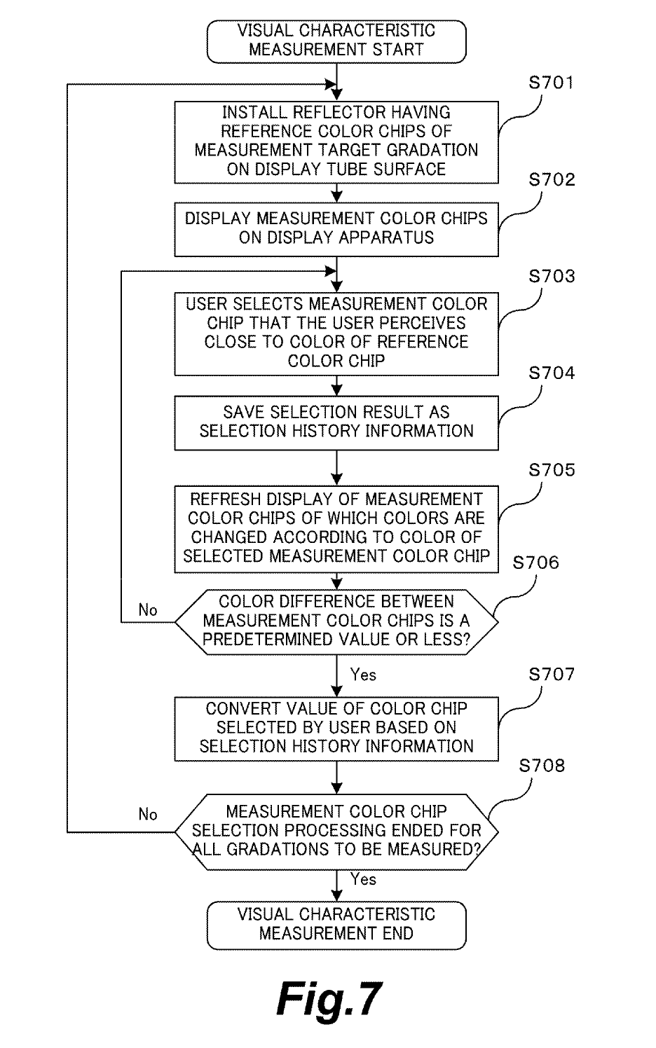

The processing flow to be a basis for the personal visual characteristic acquisition method of Example 1 has been described with reference to FIG. 3. Now the visual characteristic acquisition processing flow using the selection history information, which is characteristic processing of Example 1, will be described with reference to FIG. 7.

FIG. 7 is a flow chart depicting the processing procedure of Example 1. Each step will now be described.

The processing operations in step S701 to step S703 are the same as the processing operations in step S301 to step S303 in FIG. 3.

In step S704, the selection history record control unit 211 saves the information selected by the user in step S703 as the selection history information.

The processing in step S705 is the same as the processing in step S304.

In step S706, the visual characteristic acquisition control unit 214 determines whether the color difference between measurement color chips 105 is a threshold or less, and if a threshold or less, processing advances to step S707. If not a threshold or less, then the processing from step S703 is repeated.

In step S707, the selected color chip value conversion processing unit 212 converts the value of the measurement color chip 105 selected by the user using the selection history information recorded in step S704. The measurement color chip value conversion method in step S707 will be described later.

The processing in step S708 is the same as the processing in step S306 in FIG. 3.

An example of the selection history information that is saved in step S704 will be described with reference to a drawing.

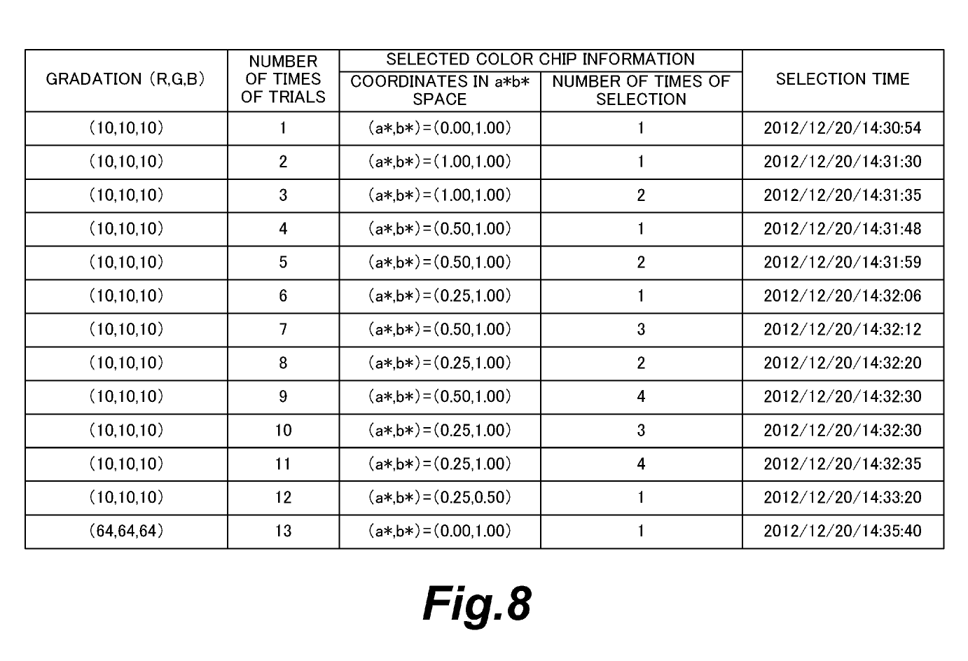

FIG. 8 is a table showing the selection history information. Information saved in the selection history information is gradation information at measurement, information on a measurement color chip selected by the user (selected color chip information), and time information when the selection was executed. The selected color chip information includes a measurement color chip value and a selection count when this measurement color chip value was selected. This information is recorded in sequence in the visual characteristic acquisition step.

The measurement color chip value conversion method executed in step S707 in the flow of Example 1 will be described next with reference to drawings. In step S707, if the user repeatedly selects a measurement color chip from a combination of two or more colors for a predetermined number of times or more, the visual characteristic of the user is calculated based on the colors included in this combination. In concrete terms, the visual characteristic of the user is calculated based on a color determined by averaging the colors included in the combination, or a color determined by weighting and adding the colors included in the combination.

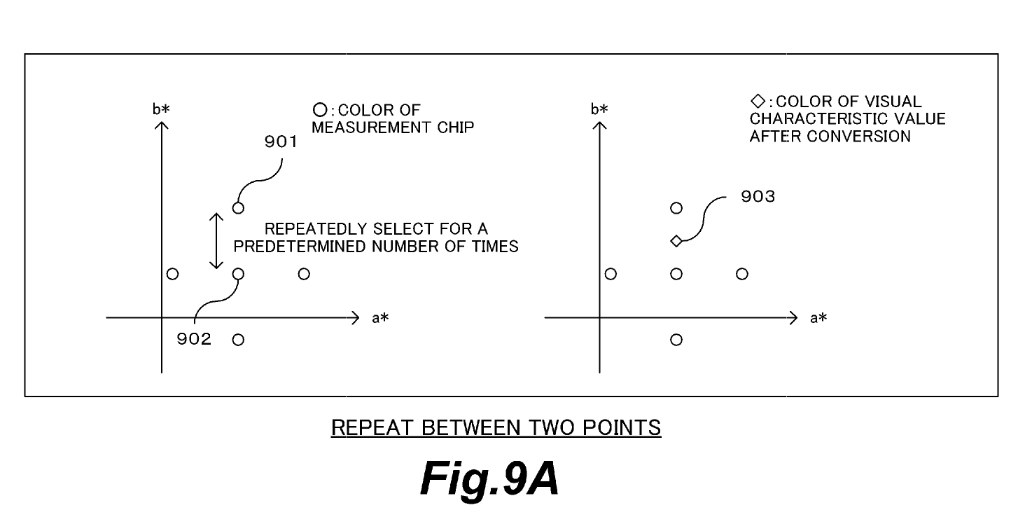

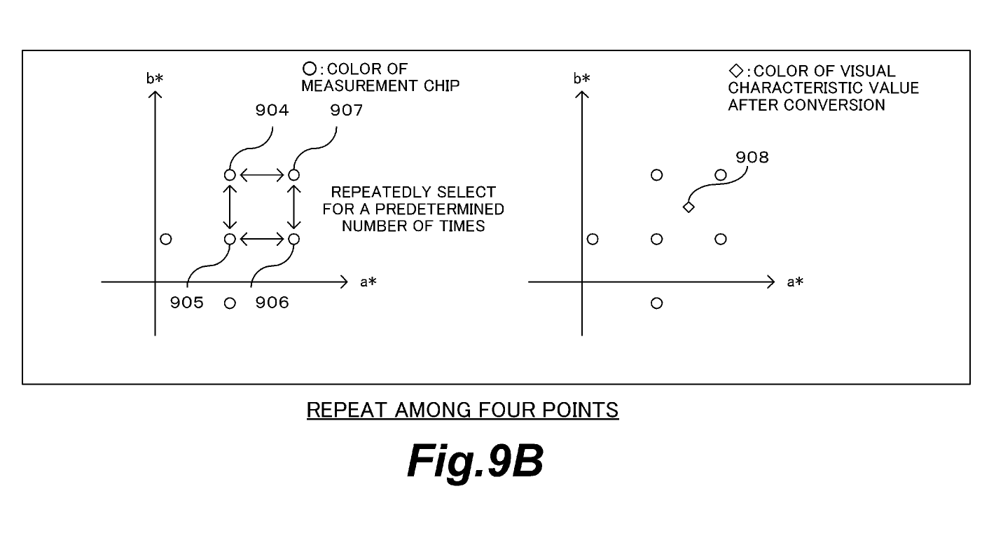

FIG. 9 are diagrams depicting a method of calculating the position indicating the visual characteristic of the user using the result of the measurement color chip 105 which the user selected.

FIG. 9A shows an example when the selection color chip values indicated by 901 and 902 are repeatedly selected for a predetermined number of times in the step of selecting the measurement color chip 105. If 901 and 902 are repeatedly selected for a predetermined number of times, it can be determined that a color located between 901 and 902 is a color closest to the visual characteristic of the user. Therefore after the user selected the measurement color chip 105, the selected color chip value conversion processing unit 212 confirms the selection history information. Then if repeated processing, as shown in FIG. 9, is detected just before selecting the measurement color chip 105 for the last time, the selected color chip value conversion processing unit 212 calculates the center of gravity position among the measurement color chips 105 as the visual characteristic value of the user. In the example in FIG. 9A, 903 indicated by ⋄ is the position of the color which indicates the visual characteristic value after conversion.

In the same manner, if the measurement color chips 105 at four points are repeatedly detected, as shown in FIG. 9B, the selected color chip value conversion unit 212 regards 908, which is the center of gravity of the four points, as the visual characteristic value after conversion.

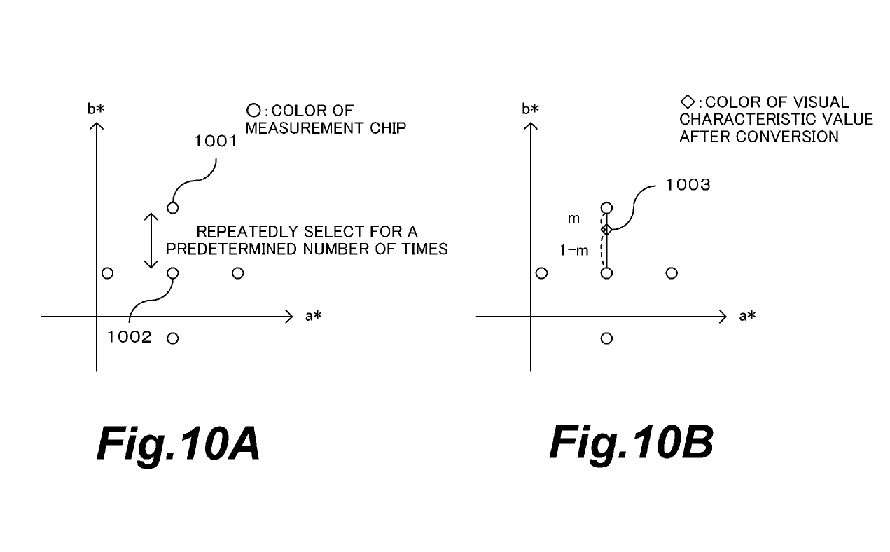

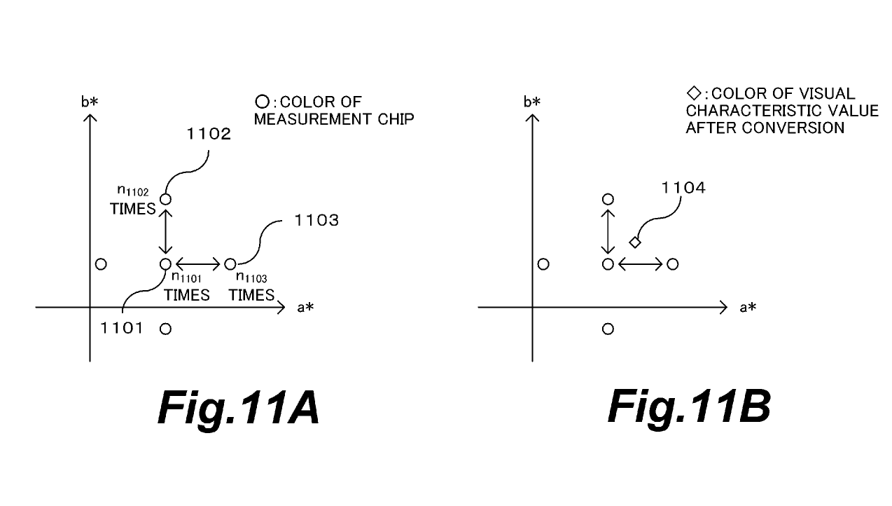

In the measurement color chip value conversion method performed in step S707, an example of another calculation method will be described with reference to FIG. 10 and FIG. 11.

FIG. 10 shows a conversion example when repeat selection is performed in the step of selecting the measurement color chip 105, just like FIG. 9. Here it is assumed that the colors indicated by 1001 and 1002 in FIG. 10 are repeatedly selected, then 1001 is selected by the user as a final result. In this case, it is presumed that the visual characteristic value of the user is in a position closest to the color of 1001, and 1003, which is the converted visual characteristic value, is calculated by the following expressions.

a*₁₀₀₃=a*₁₀₀₁×m+a*₁₀₀₂×(1−m)

b*₁₀₀₃=b*₁₀₀₁×m+b*₁₀₀₂×(1−m) [Math. 1]

In the above expressions, m denotes a weight to calculate the visual characteristic value, and is a value greater than 0.5 and smaller than 1.0. By the above expressions, a value closer to 1001 is calculated, as indicated by 1003 in FIG. 10. In other words, a color, of which timing selected by the user is late, is weighted with a weight greater than the weights of the other colors, and the visual characteristic value is calculated based on the color determined by weighting and adding the colors in this way.

FIG. 11 shows a case when a plurality of measurement color chips 105 are repeatedly selected, and is an example of calculating a converted visual characteristic value by weighting each measurement color chip 105 based on the number of times of selection.

FIG. 11 is an example of the visual characteristic value calculation method when the measurement color chips 105 at three points, indicated by 1101, 1102 and 1103, are repeatedly selected. The converted visual characteristic is calculated using the following expressions where n denotes the selection count.

$$a {*}_{1104} = \begin{matrix}a {*}_{1101} \times {n}_{1101} + a {*}_{1102} \times {n}_{1102} + a {*}_{1103} \times {n}_{1103} \\ ( {n}_{1101} + {n}_{1102} + {n}_{1103} )\end{matrix} \\ b {*}_{1104} = \frac{b {*}_{1101} \times {n}_{1101} + b {*}_{1102} \times {n}_{1102} + b {*}_{1103} \times {n}_{1103}}{( {n}_{1101} + {n}_{1102} + {n}_{1103} )} \qquad [ Math . 2 ]$$

Since a color, close to the position of the measurement color chip 105 of which selection count is high, is calculated as the converted visual characteristic value, the visual characteristic value can be calculated based on a more accurate estimation.

As described above, the visual characteristic value of the user can be acquired at higher precision by converting the measurement color chip value, selected by the user, into a different color using repeat selection information written in the selection history information.

The visual characteristic value calculation method described in Example 1 is an example, and the present invention is not limited to this.

### Example 2

Example 1 is an example of converting the measurement color chip value selected by the user at high precision using the selection history information. Example 2 shows an example of determining whether a desired precision can be acquired by calculating the visual characteristic value using the selection history information, and interrupting acquisition of the visual characteristic if the desired precision can be acquired.

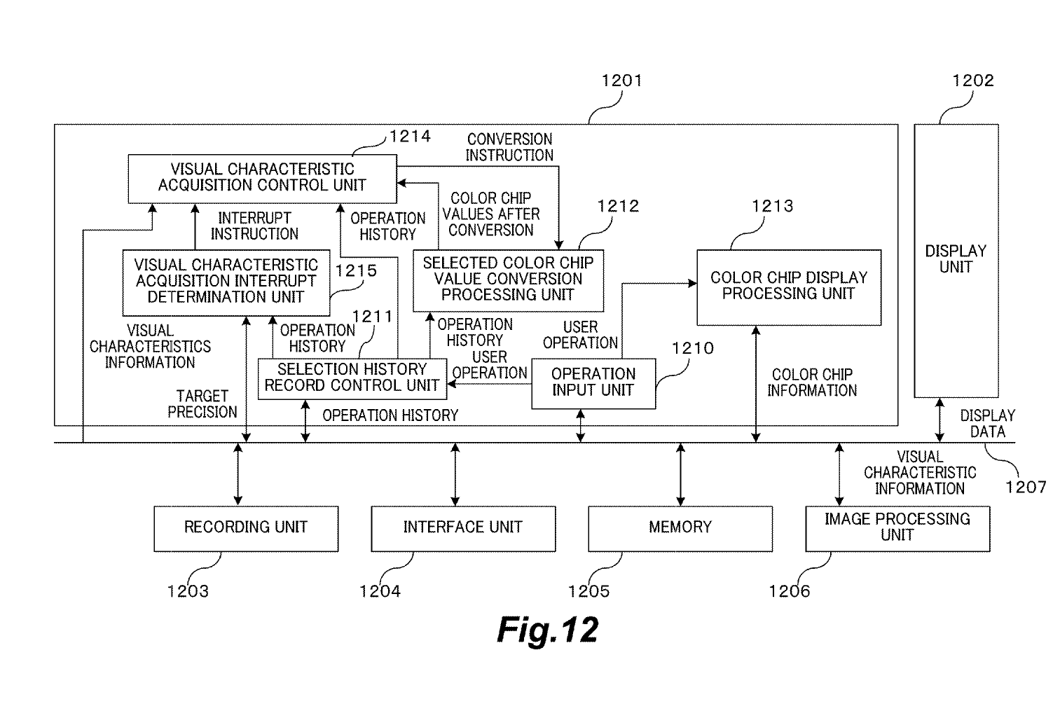

The functional blocks of Example 2 will be described with reference to FIG. 12. In FIGS. 12, 1201 to 1207 and 1210 to 1213 have the same functions as 201 to 207 and 210 to 213 in FIG. 2, hence detailed description thereof will be omitted.

A visual characteristic acquisition control unit 1214 in FIG. 12 performs control for acquiring a visual characteristic value of the user using the selection history information of the measurement color chip 105 recorded in a selection history record control unit 1211.

A visual characteristic acquisition interrupt determination unit 1215 in FIG. 12 performs processing to determine whether a visual characteristic value, which specifies the target precision, can be acquired or not by calculating the visual characteristic value using the selection history information. For the target precision value, a value saved in the recording unit 1203 in advance or a value inputted by the user is used.

Now a flow of Example 2 will be described with reference to a drawing.

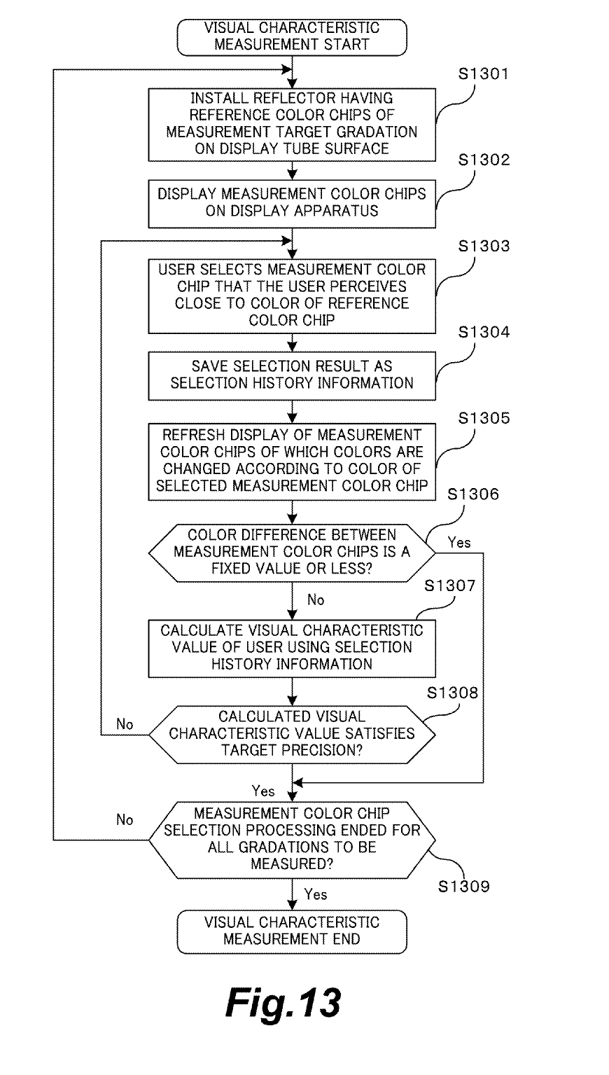

FIG. 13 is a flow chart depicting the processing procedure of Example 2. Each step will now be described.

The processing operations in step S1301 to step S1305 are the same as the processing operations in step S701 to step S705.

In step S1306, the visual characteristic acquisition control unit 1214 determines whether the color difference between the measurement color chips 105 is a threshold or less, and if a threshold or less, processing advances to step S1309. If not a threshold or less, processing advances to step S1307.

The processing in step S1307 is the same as the processing in step S707 in FIG. 7.

In step S1308, the visual characteristic acquisition interrupt determination unit 1215 determines whether the visual characteristic value calculated in step S1307 satisfies the target precision. If target precision is satisfied, processing advances to step S1309. If target precision is not satisfied, processing from step S1303 is restarted.

The processing in step S1309 is the same as the processing in step S708 in FIG. 7.

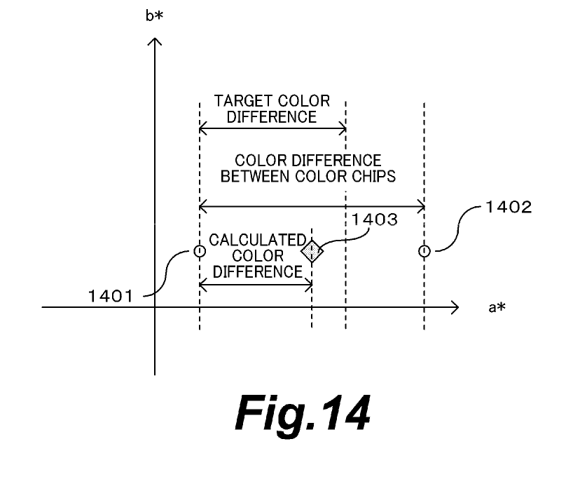

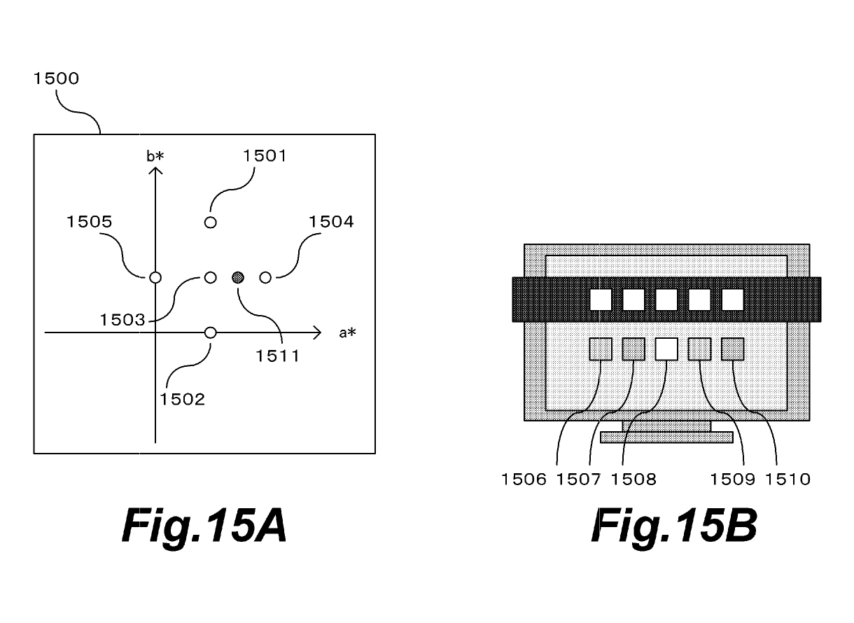

The relationship between the target precision and the calculated visual characteristic precision will be described with reference to FIG. 14.

FIG. 14 is a schematic diagram depicting the relationship between the calculated precision based on the selection history and the target precision. In FIG. 14, the target precision is described as the target color difference. Improving the target precision means decreasing the difference between the true visual characteristic value of the user and the measured visual characteristic value. In the visual characteristic measurement, the visual characteristic value can be measured at higher precision by decreasing the color difference between the measurement color chips 105, hence improving precision means decreasing the color difference.

1401 and 1402 in FIG. 14 indicate the measurement color chips 105, and the color distance between 1401 and 1402 is the inter-color chip color difference. Here if the user selected 1401 and 1402 repeatedly according to the selection history, the visual characteristic is calculated as 1403. The calculated visual characteristic value has a precision of the calculated color difference which is indicated as the distance between 1401 and 1403. If the calculated color difference is smaller than the target color difference, the visual characteristic value of the user is determined as 1403, whereby the visual characteristic acquisition processing by the user can be interrupted while satisfying the target color difference.

As described above, according to Example 2, if the visual characteristic value that satisfies the target precision can be acquired based on the selection history information thus far, the visual characteristic value can be acquired by calculation based on the selection history information after the visual characteristic acquisition processing is interrupted. By Example 2, the visual characteristic that satisfies the target precision can be acquired with decreasing the user load.

### Example 3

Example 3 is an example of the arithmetic processor of the present invention which performs processing to acquire a visual characteristic, where the user compares the reference color chips of the printed matter and the measurement color chips of the display apparatus. Then when the visual characteristic is acquired by the user repeatedly selecting a measurement color chip of which color is close to the reference color chip, the display colors of the measurement color chips are changed if the similar measurement color chip has been repeatedly selected in the history of the selection processing.

Example 3 will now be described with reference to a drawing.

The composing elements of Example 3 are the same as those in FIG. 1, which were described in Example 1, therefore description thereof will be omitted.

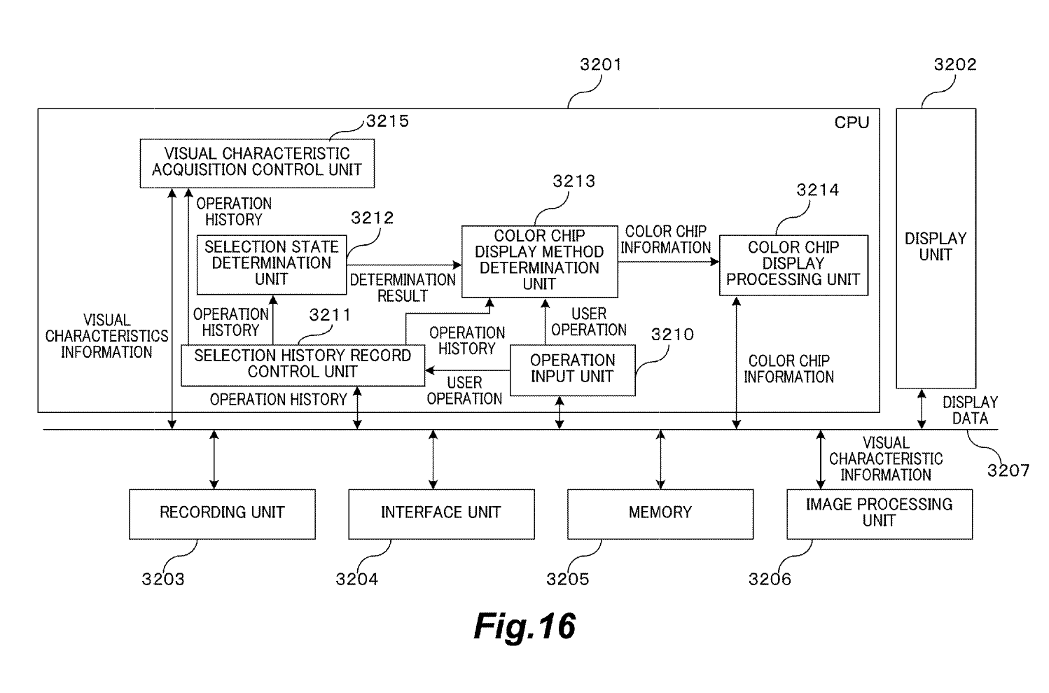

FIG. 16 is a diagram depicting the functional blocks of Example 3. Each functional block will now be described.

3201 in FIG. 16 denotes a CPU (Central Processing Unit) which performs various controls. In the CPU 3201, control upon acquiring the visual characteristic and a later mentioned processing to change the color chip display method are executed. Other controls such as reading various data, are also executed by the CPU 3201.

3202 to 3211 in FIG. 16 are the same as 202 to 211 in FIG. 2, which were described in Example 1, hence description thereof will be omitted.

A selection state determination unit 3212 in FIG. 16 determines whether the user is having difficulty in selecting a color chip based on the selection history information when the visual characteristic of the use is acquired. If it is determined that the color chips selected by the user are not converged to one, the selection state determination unit 3212 determines that the user is having difficulty in selecting the color chip. In concrete terms, the selection state determination unit 3212 determines that the color chips are not converged when the user repeatedly selects a same plurality of color chips, or when it takes a predetermined time or longer for the user to select a color chip. Details will be described later.

A color chip display method determination unit 3213 determines a display method to display the measurement color chips 105 on the display apparatus 102 in FIG. 1, based on the determination result by the selection state determination unit 3212. “Determining the display method” here means determining the mode of display, such as number, size, color and the like of the measurement color chips 105 to be displayed.

A color chip display processing unit 3214 in FIG. 16 performs processing to actually display the measurement color chips 105 on the display apparatus 102. In concrete terms, the color chip display processing unit 3214 writes the measurement color chips 105 to a frame buffer created in the memory 3205 according to the display method to display the measurement color chips 105 determined by the color chip display method determination unit 3213.

A visual characteristic acquisition control unit 3215 in FIG. 16 performs control to acquire the visual characteristic value of the user using the selection history information of the measurement color chips 105 recorded in the selection history record control unit 3211.

The visual characteristic measurement method of Example 3 will now be described with reference to the drawings.

The processing flow to be a basis for the visual characteristic acquisition method of Example 3 will be described with reference to the flow chart in FIG. 3. The processing flow in FIG. 3 is controlled by the visual characteristic acquisition control unit 3215 in FIG. 16. Detailed description will be omitted for processing of which content is similar to the description in Example 1.

The processing in step S301 is as described in Example 1.

The processing in step S302 is the same as Example 1, except that the color chip display processing unit 3214 executes this processing.

In step S303, the user selects a measurement color chip 105 which satisfies a predetermined condition with respect to the reference color chip 104, out of the measurement color chips 105 which are displayed. Here it is assumed that the measurement color chip which satisfies a predetermined condition is a color chip of which color is closest to that of the reference color chip 104. In other words, the user performs processing of selecting a measurement color chip 105 of which color is perceived to be closest to that of the reference color chip 104.

In step S304, the color chip display processing unit 3214 refreshes the display of the measurement color chips 105 of which colors are changed based on the colors of the measurement color chip 105 selected by the user in step S303. The colors of the measurement color chips 105 are determined by the color chip display method determination unit 3213.

The processing operations in step S305 and step S306 are as described in Example 1, except that the visual characteristic acquisition control unit 3215 executes these processing operations.

The processing operations executed in step S303 and step S304 are as described in detail in Example 1 with reference to FIG. 4, therefore description thereof will be omitted.

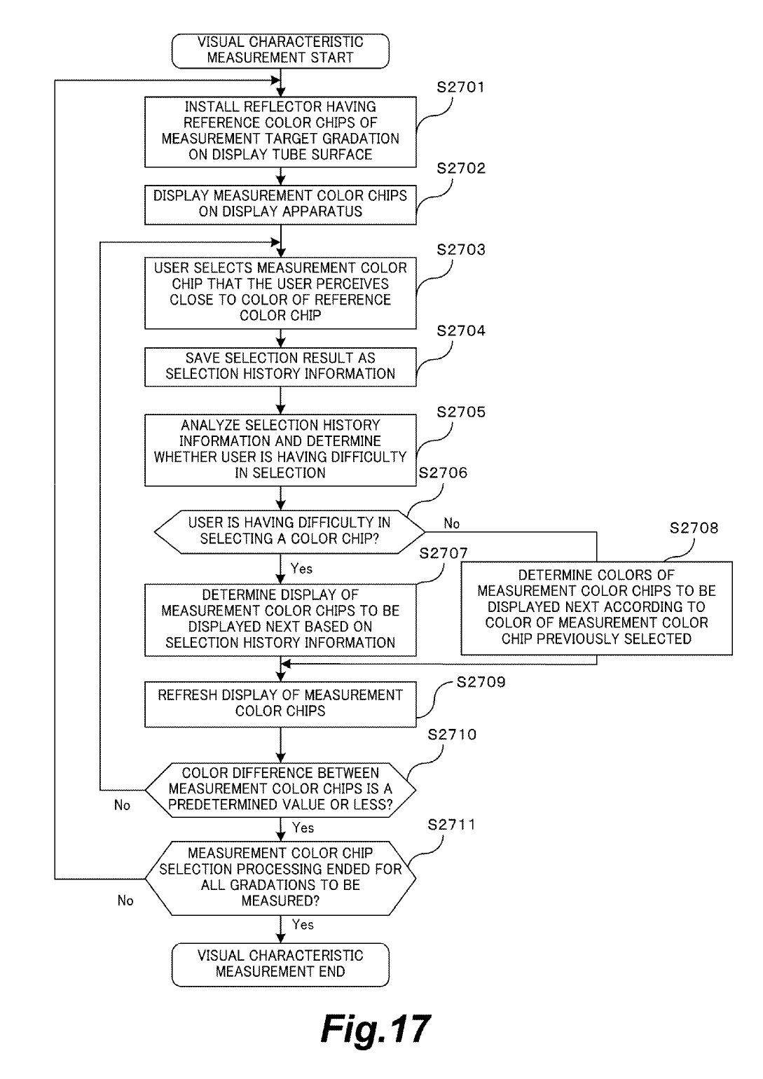

The flow of the visual characteristic acquisition processing using the selection history information, which is a characteristic processing of Example 3, will now be described with reference to FIG. 17.

FIG. 17 is a flow chart depicting the processing procedure of Example 3. Each step will now be described.

Processing operations in step S2701 to step S2703 are the same as the processing operations in step S301 to step S303 in FIG. 3.

In step S2704, the selection history record control unit 3211 saves the information selected by the user in step S2703 as the selection history information.

In step S2705, the selection state determination unit 3212 determines whether the user is having difficulty in selecting a measurement chip 105, based on the selection history information recorded in step S2704. If it is determined that the user is having difficulty (step S2706: YES), processing advances to step S2707. If it is determined that the user is not having difficulty (step S2706: NO), processing advances to step S2708.

In step S2707, the color chip display method determination unit 3213 determines a display method to display the measurement color chips 105 based on the selection history information, so as to avoid the state of the user having difficulty in selecting a color chip.

In step S2708, colors of the measurement color chips 105, to be displayed next time, are determined using a processing similar to step S304 in FIG. 3, based on the colors of the measurement color chips selected in step S2703.

In step S2709, the display of the measurement color chips 105 is refreshed according to the colors of the color chips determined in step S2707 or step S2708.

In step S2710, the visual characteristic acquisition control unit 3215 determines whether the color difference between the measurement color chips 105 is a threshold or less, and if a threshold or less, processing advances to step S2711. If not a threshold or less, processing from step S2703 is repeated.

The processing in step S2711 is the same as the processing in step S306 in FIG. 3.

An example of the selection history information saved in step S2704 is as described in Example 1 with reference to FIG. 8, therefore description thereof will be omitted.

A method for detecting a state where the user is having difficulty in selecting a measurement color chip 105 in the visual characteristic measurement step in Example 3 will now be described with reference to the drawings. In the following description, a color is expressed by a point in the L*a*b* space. For example, “color at a center of gravity of three colors” refers to a color corresponding to a point which is located at a center of gravity position of the points corresponding to the three colors in the L*a*b* space. In Example 3, the L*a*b* space will be described as an example of the color space, but the present invention is not limited to this.

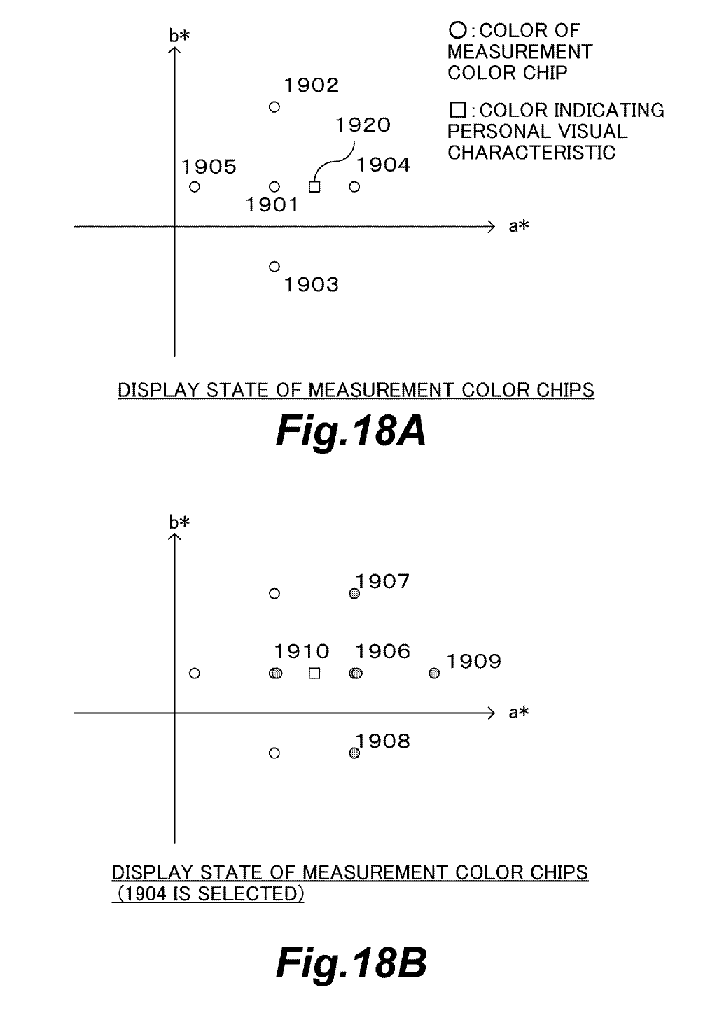

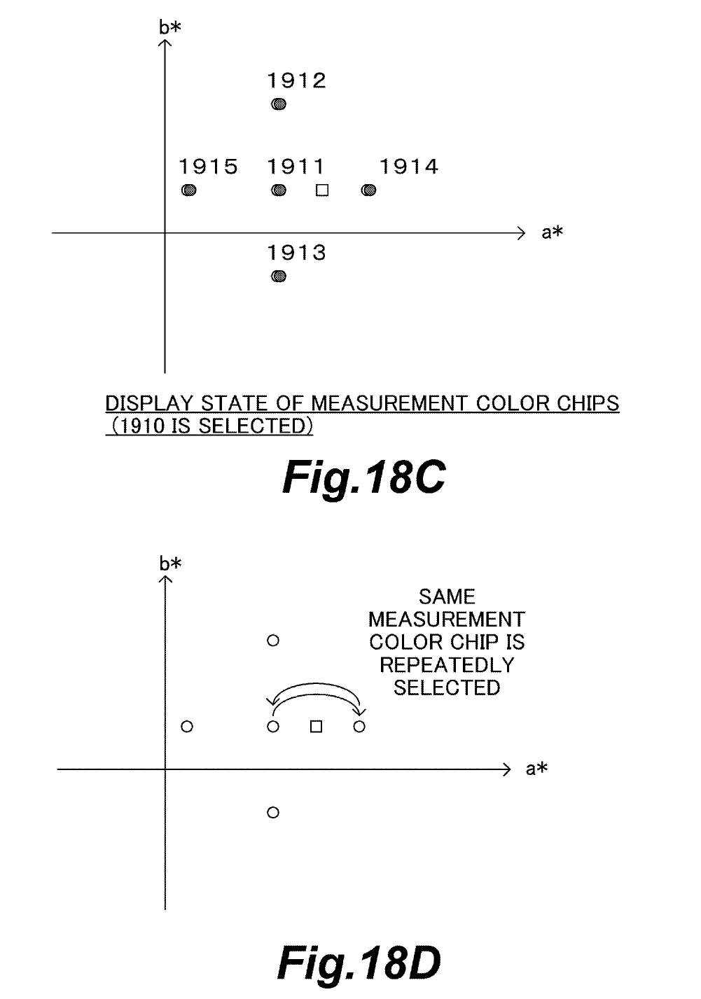

FIG. 18 shows an example when the personal visual characteristic is located approximately at the center between two measurement color chips 105, and in this state, the two points are repeatedly selected and the visual characteristic is not converged, indicating that the user is having difficulty in selecting a color chip.

FIG. 18A is an example when a certain combination of measurement color chips 105 is displayed. 1901 to 1905 indicate the colors of the measurement color chips 105. It is assumed that 1920, indicated by □ in FIG. 18A, is a value representing the visual characteristic of the user. 1920 has a value located approximately at the center between 1901 and 1904 in the measurement color chips 105.

Here it is assumed that the user selected 1904 as a color closet to the reference color chip 104. In this case, the display of the measurement color chips 105 is refreshed as shown in FIG. 18B. 1906 to 1910 indicate the colors of the measurement color chips 105, where 1906 and 1904 are the same colors, and 1910 and 1901 are the same colors. In the state of FIG. 18B, the visual characteristic of the user is located between 1906 and 1910, therefore the user probably selects 1906 or 1910 as a measurement color chip of which color is close to the reference color chip.

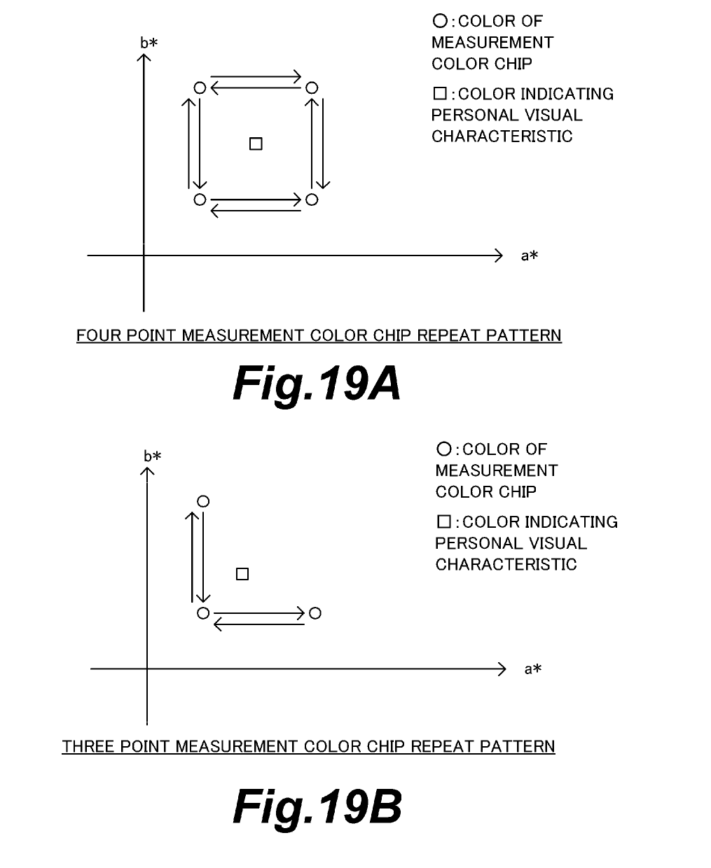

If the user selects 1910, the display state changes to the state shown in FIG. 18C. 1911 to 1915 are positions indicating the refreshed colors of the measurement color chips 105. 1911 to 1915 are the same colors as 1901 and 1905 shown in FIG. 18A. In other words, the display state reverted back to the state in FIG. 18A. Thus if a color indicating the visual characteristic is located approximately at the center between two points, for example, these two points may be selected alternately, then the selection processing is not converged, and the user has difficulty in selecting a color chip. The occurrence of this state of the user having difficulty in selecting a color chip is not limited to two points, but as shown in FIG. 19, the selection processing may not be converged when a plurality of points are repeatedly selected.

In step S2705 in FIG. 17, the selection state determination unit 3212 detects that a same measurement color chip 105 is repeatedly selected for a plurality of times, as shown in FIG. 18, whereby it is determined whether the user is having difficulty in selecting a color chip. In concrete terms, the selection state determination unit 3212 determines that the user is having difficulty in selecting a color chip if a number of times when a predetermined plurality of certain color chips are repeatedly selected exceeds a predetermined threshold in the selection history information shown in FIG. 8. By referring to the color chip selection count in the selection history information, such a state of the user can be detected.

Now a method for changing the display method to display the measurement color chips 105 in the case when the user is having difficulty in selecting a color chip will be described with reference to the drawings. In Example 3, a color chip of a central color, which is determined based on a plurality of certain color chips repeatedly selected by the user, and one or more vicinity color(s) having a color difference from the color chip are determined as the color chips to be displayed on the display apparatus next time.

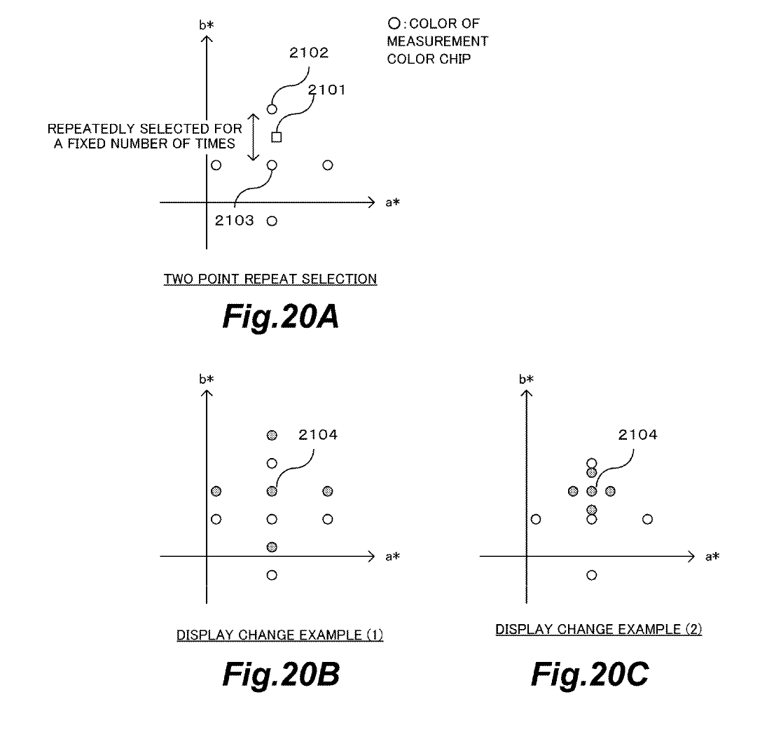

FIGS. 20A to 20C are diagrams depicting a refresh of the display of the measurement color chips 105 when two points are repeatedly selected. In FIG. 20A, 2101 indicates the visual characteristic of the user, and the measurement color chips 105 indicated by 2102 and 2103 are the repeatedly selected measurement color chips 105.

If the state shown in FIG. 20A is generated, the display of the measurement color chips 105 is refreshed, as shown in FIG. 20B or FIG. 20C, whereby the state of the user selecting the same measurement color chips repeatedly can be cleared.

FIG. 20B shows a method where it is assumed that the visual characteristic value of the user exists at a center between the repeatedly selected measurement color chips 105, and the colors of the measurement color chips 105, to be displayed next time, are determined at positions centering around 2104, which is the center of the gravity position of 2101 and 2012. In other words, the central color of the measurement color chips to be displayed next is determined by averaging the repeatedly selected measurement color chips 105. FIG. 20C shows a method where 2104 is set as the center, and when the display is refreshed, the color difference between the measurement color chips 105 is decreased. If the central color of previously displayed measurement color chips is the same as the central color determined based on the measurement color chips which the user selected this time, the color difference between the central color of the measurement color chips to be displayed next time and a vicinity color thereof should be set to be smaller than the last time. By making the central color located at the center of gravity position between the repeatedly selected measurement color chips 105 to be the central color chip of the refreshed measurement color chips 105, as shown in FIG. 20B or FIG. 20C, a method for avoiding the state of the user having difficulty in selecting a color chip can be provided. In this example, a color at the center of gravity position of the two measurement color chips, that is, the color determined by averaging the colors of the two measurement color chips, is determined as the central color of the colors of the measurement color chips to be displayed next time, but weighted averaging may be used for the averaging operation as well.

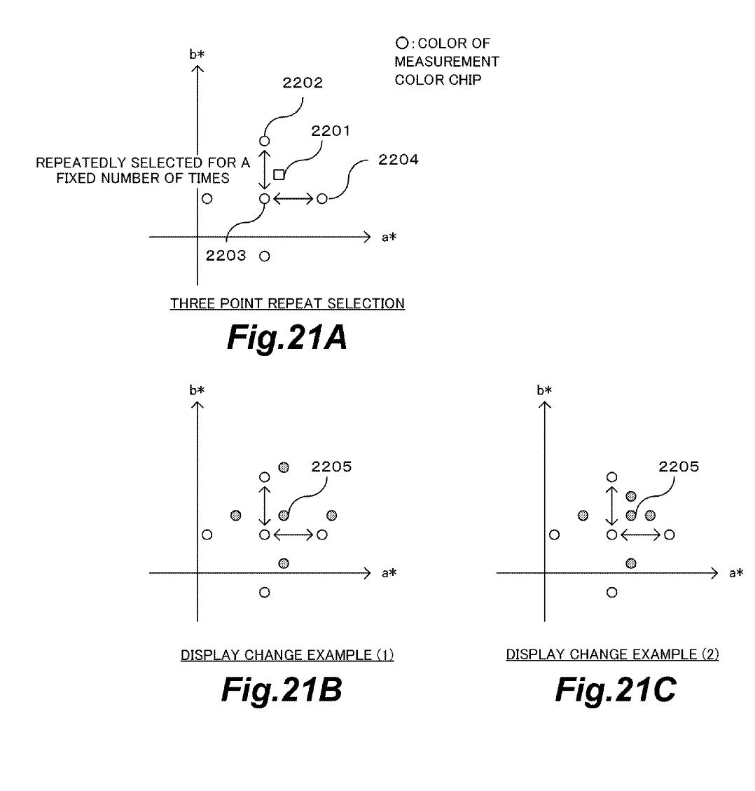

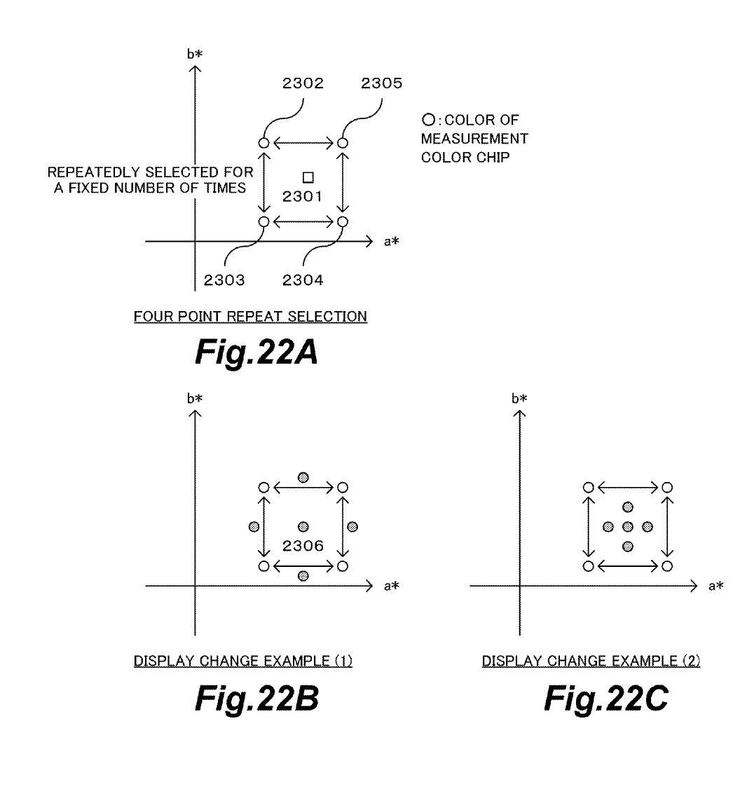

FIG. 21 shows an example of a refresh of the display of the measurement color chips 105 when three points are repeatedly selected, and FIG. 22 shows an example of a refresh of the display of the measurement color chips 105 when four points are repeatedly selected.

In FIG. 21A, 2201 indicates a position representing the visual characteristic of the user, and the measurement color chips 105 indicated by 2202, 2203 and 2204 were repeatedly selected. If this state is generated, the colors of the measurement color chips 105 are determined regarding the center of gravity position of the selected measurement color chips 105 as the center, just like FIG. 20. FIG. 21B shows an example when the color difference between the measurement colors chips 105 is constant, and FIG. 21C shows an example when the color difference between the measurement color chips 105 is different depending on the color. In the case of FIG. 21C, it is determined that the visual characteristic value of the user is located in the upper right direction of the center of gravity position 2205, because of the repeated state of selection. Therefore in this example, the color difference from the color at the center of gravity position is decreased for a measurement color chip 105 located in the upper right direction of the center of gravity position, and the color difference from the color at the center of gravity position is increased for a measurement color chip 105 located in the lower left direction of the center of the gravity position. In other words, in FIG. 21C, the color difference between the central color and a vicinity color is set to be smaller as the vicinity color is presumed to be closer to the visual characteristic of the user.

In FIG. 22A, 2301 indicates a position representing the visual characteristic of the user, and a case where processing of repeatedly selecting the measurement color chips 105 indicated by 2302, 2303, 2304 and 2305 is performed. If this state is generated, the colors of the measurement color chips 105 to be displayed next are determined regarding the center of gravity position of the selected measurement color chips 105 as the center, as shown in FIG. 21B and FIG. 21C.

In Example 3, the user selects a measurement color chip of which color is closest to the reference color, out of a plurality of different measurement color chips displayed on the display apparatus, and the selected measurement color chip and one or more measurement color chip having a color difference from the measurement color chip are displayed on the display apparatus next time, and this processing is repeated. If it is determined that measurement color chips selected by the user are not converged to one based on the history information, which is information on the result of repeating for a plurality of times the determination of measurement color chips and selection by the user, the measurement color chips to be displayed on the display apparatus are changed. In other words, it is determined that the user is having difficulty in selecting a color chip by detecting that the same measurement color chip 105 was repeatedly selected. Then the display mode of the measurement color chips 105 is changed based on the repeat state, whereby the state of the user having difficulty in selecting a color chip can be avoided. As a consequence, time required for acquiring the visual characteristic can be decreased, and the visual characteristic of the user can be efficiently acquired.

The method for detecting the state of the user having difficulty in selecting a color chip and the display method to display the measurement color chips 105 described in Example 3 are examples, and the present invention is not limited to these methods.

### Example 4

In Example 3, it is detected that the user is having difficulty in selecting a color chip when the same measurement color chips 105 are repeatedly selected for a predetermined number of times or more, and the colors of the measurement color chips 105 to be displayed are changed according to the repeat state. In Example 4, it is detected that the user is having difficulty in selecting a color chip when the user does not perform selection processing for a predetermined period when a measurement color chip 105 is selected, and the display method to display the measurement color chips 105 is changed.

The functional blocks and the general processing flow of the arithmetic processor, to acquire the visual characteristic according to Example 4 are the same as Example 3, except for the method for determining whether the user is having difficulty in selecting a color chip, hence detailed description thereof will be omitted.

A method for determining whether the user is having difficulty in selecting a color chip according to Example 4 will be described. This determination processing is the processing executed in step S2705 in FIG. 17.

As shown in FIG. 8, the selection time, when a measurement color chip 105 is selected, is recorded in the selection history information. If the elapsed time, from the time when a measurement color chip 105 was selected last time, becomes a threshold or more in step S2705, and the next measurement color chip 105 is not selected, it is determined that the user is having difficulty in selecting a color chip. If a next measurement color chip 105 is not selected even after a long time has elapsed from the last selection of a measurement color chip 105, it is presumed that the user is having difficulty in selecting a measurement color chip 105, since all the measurement color chips 105 look similar to the reference color chip 104.

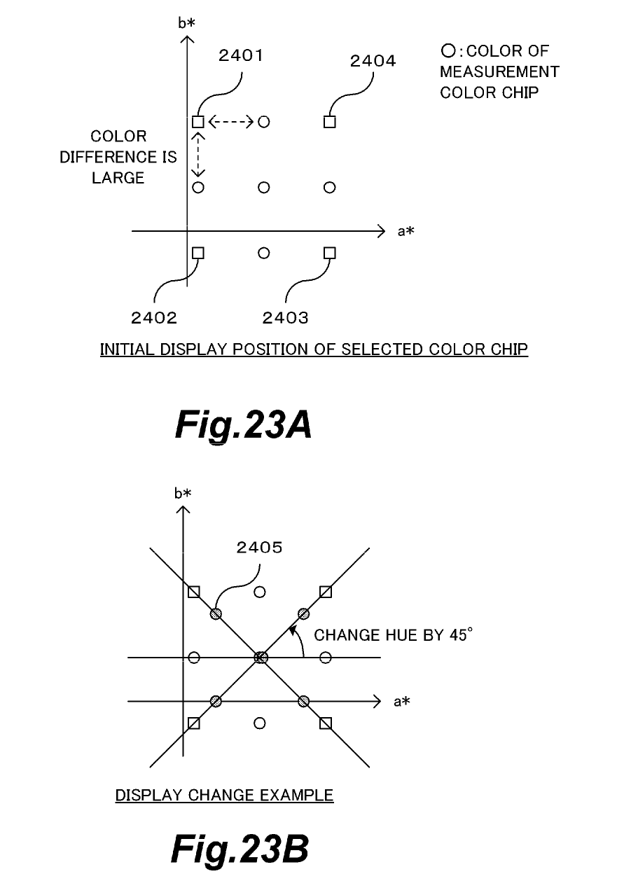

The method for determining the colors of the measurement color chips 105, to be refreshed when the user does not execute the selection processing, will be described with reference to FIG. 23.

FIG. 23A shows a certain display state of the measurement color chips 105. ◯ in FIG. 23 indicates a position of a measurement color chip 105.

In the state of FIG. 23A, if the user does not execute the selection processing for a predetermined time, the selection state determination unit 3212 determines that no color of the measurement color chips 105 looks similar to the reference color chip 104 for the user, and the user is having difficulty in selection. In this case, it is presumed that a color indicating the visual characteristic of the user is located in a position which has a predetermined color difference or more from all the measurement color chips 105 in the vicinity, such as 2401 to 2404 in FIG. 23. Therefore as shown in FIG. 23B, the colors of the measurement color chips to be displayed next time are determined by changing the hue of the colors of the previously displayed measurement color chips. In the example in FIG. 23B, hue is changed by 45°. As shown in FIG. 23B, by displaying the measurement color chips 105 using a hue that is different from that of the previously displayed measurement color chips 105, selection by the user can be facilitated, and the state of the user having difficulty in selecting a color chip can be avoided.

As described above, in Example 4, the time information when the selection was performed in the selection history information is utilized, and if the user does not perform the selection processing for a predetermined time, it is determined that the user is having difficulty in selecting a color chip, and the display method to display the color chips is changed. By displaying the new measurement color chips 105 using different colors from the current measurement color chips 105, a visual characteristic acquisition method, which avoids the state of the user having difficulty in selecting a color chip, can be provided, and the time to acquire the visual characteristic can be shortened.

The method for detecting a state of the user having difficulty in selecting a color chip and the display method to display the measurement color chips 105 described in Example 4 are examples, and the present invention is not limited to these methods.

### Example 5

In Examples 3 and 4, a visual characteristic acquisition method, using the comparison reflector 103 irradiated by the high color rendering fluorescent light 101 in FIG. 1 as the reference color chip 104, was described. In Example 5, the reference color chips 104 are displayed on the display apparatus 102, just like the measurement color chips 105. By displaying the reference color chips 104 on the display apparatus 102, the size and number of color chips can be easily changed, and the state of the user having difficulty in selecting a color chip can be more effectively detected, and the state of the user having difficulty in selecting a color can be avoided.

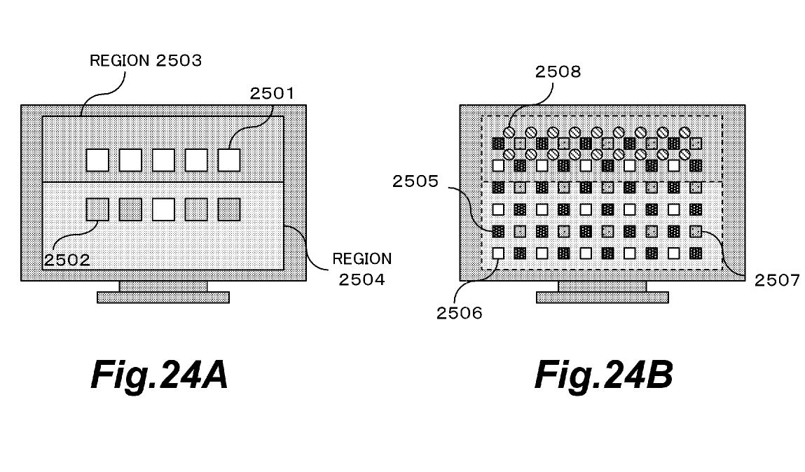

FIG. 24 shows a display apparatus 102, which is used in Example 5 and depicted in FIG. 1. 2501 in FIG. 24 indicates a reference color chip 104 and 2502 indicates a measurement color chip 105. The display apparatus 102 shown in FIG. 24 is divided into two regions, each of which has a different backlight. In a first region 2503 in which the reference color chips 104 are displayed, a backlight is included which has a broadband emission spectrum, just like the high color rendering fluorescent light 101. A second region 2504 is a normal backlight region, and the measurement color chips 105 are displayed in the region indicated by 2504. Here it is assumed that the second region 2504 includes a backlight of which emission spectrum is narrower than the first region 2503.

The backlight configuration of the region 2503 and the region 2504 will now be described in detail. 2505 to 2507 in FIG. 24 are schematic diagrams depicting LEDs constituting the LED backlight. 2505 indicates a green LED, 2506 indicates a red LED, and 2507 indicates a blue LED. In the first region 2503, a white LED, indicated by 2508, is also included in order to implement a broadband emission spectrum. When the visual characteristic is acquired, not only the RGB-LEDs indicated by 2505 to 2507, but the white LEDs indicated by 2508 as well turn ON, whereby a backlight having a broadband emission spectrum, which has a predetermined value or higher spectrum over a broad wavelength region, is implemented. In normal operation when a visual characteristic is not acquired, RGB-LEDs indicated by 2505 to 2507 turn ON in the first region 2503, just like the second region 2504, so as to implement a backlight having a narrowband emission spectrum. Thus in the first region 2503, emission can be switched between a broadband emission spectrum and a narrowband emission spectrum, depending on whether the state is acquiring a visual characteristic or in normal usage. A light source having a characteristic in which the spectral radiance changes at a relatively gradual pace generates little difference in a personal visual characteristic, therefore the color chips displayed in the region 2503 having such a backlight can be used as the reference color chips 104.

In FIG. 24, the backlight to display the reference color chips 104 is implemented by increasing the number of LEDs, but a CCFL may be disposed instead. In this case, the LEDs are turned ON and the CCFL is turned OFF during normal usage, whereby the backlight performs uniform emission in all the display areas. When the visual characteristic is measured, the LEDs are turned OFF only in the region 2503, and the CCFL is turned ON, whereby the backlight emits the broadband spectrum only for a region 2503 where the reference color chips are displayed.

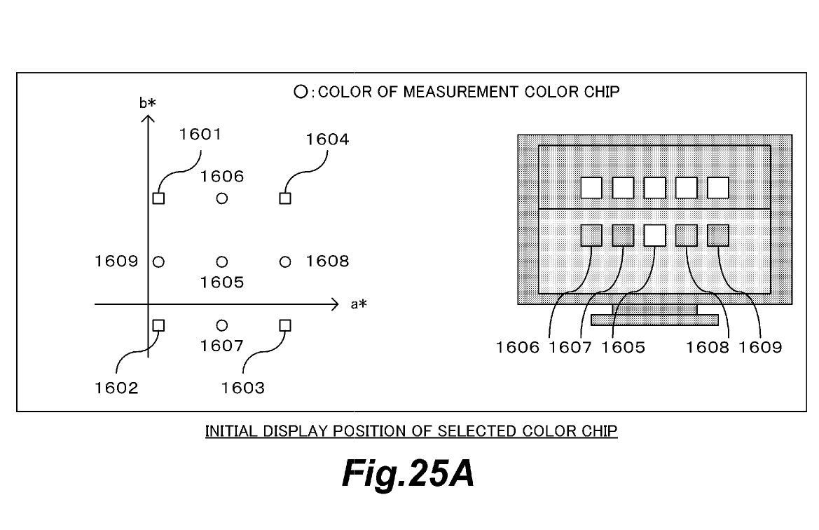

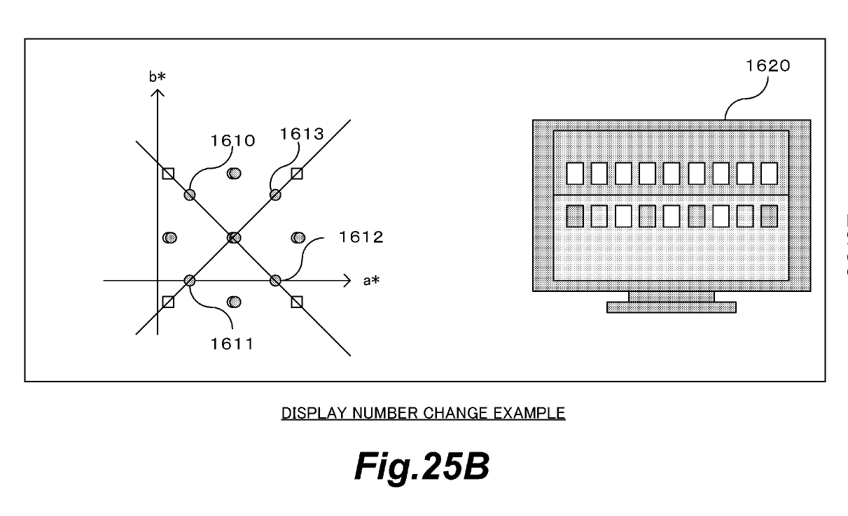

A modification of the measurement color chips 105 in Example 5 will now be described with reference to FIG. 25. FIG. 25A shows a state of displaying measurement color chips 105. Colors indicated by 1605 to 1609 are the measurement color chips 105. If the user does not perform selection processing for a predetermined time in this display state, it can be determined that all the measurement color chips 105 are different from the color of the reference color chip 104. In this case, it can be presumed that the visual characteristic of the user is located in such a position as 1601 to 1604 indicated in FIG. 25A.

If it is detected that the user is having difficulty in selecting a color chip in the state of FIG. 25A, the measurement color chips 105 are displayed as shown in FIG. 25B in Example 5. FIG. 25B shows a method for additionally displaying the measurement color chips 105 of which hue is changed, in addition to the originally displayed measurement color chips 105. 1620 shows in a schematic view this display state of the measurement color chips 105. By changing a number of measurement color chips 105 which are simultaneously displayed when the user is having difficulty in selecting a color chip, the state of the user having difficulty in selecting a color chip can be avoided. Here an example of displaying both the measurement color chips of which hue is changed and the measurement color chips before this change was described, but only the measurement color chips of which hue is changed may be displayed. As a method of changing the hue, an example of rotating 45° in the L*a*b* space was described, but the method for changing hue is not limited to this.

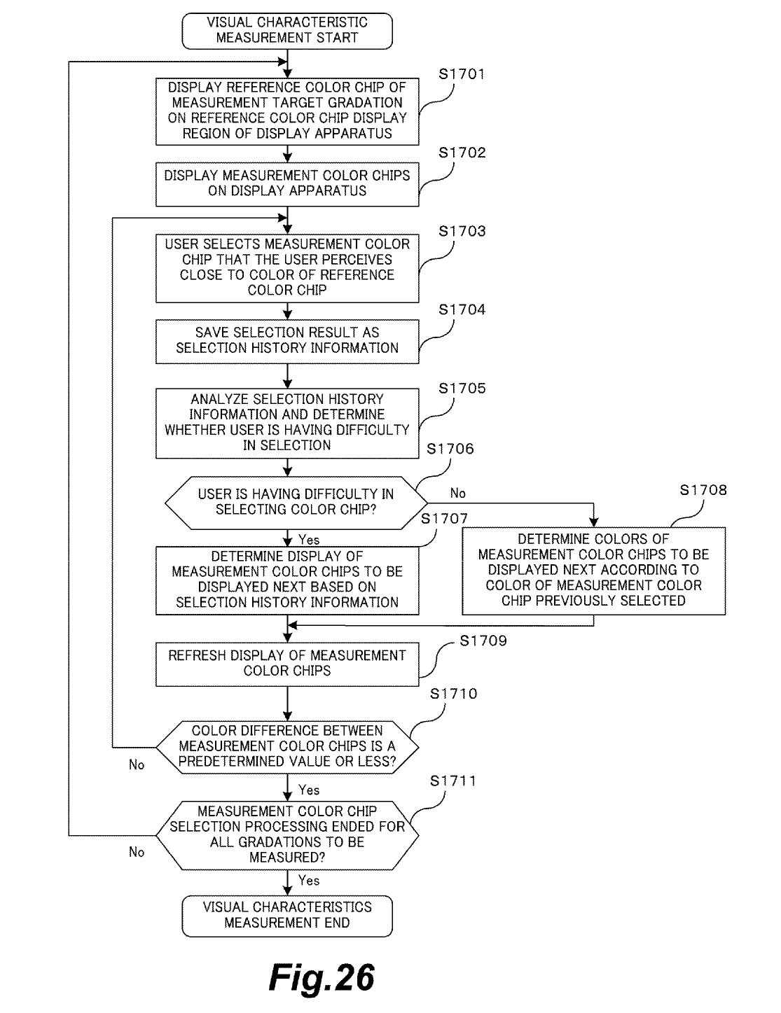

FIG. 26 shows a flow chart depicting the processing of Example 5.

In step S1701, the visual characteristic acquisition control unit 3215 displays the reference color chips in the region 2503 (region where reference color chips can be displayed) of the display apparatus.

The processing operations from step S1702 to step S1711 are processing operations corresponding to step S2702 to step S2711 in FIG. 17. The measurement color chip determination processing, based on the selection history information in step S1707, can include a change in size and number of reference color chips and the measurement color chips, as mentioned above.

In Example 5, the size and number of measurement color chips 105 can be changed by displaying the reference color chips 104 on the display apparatus 102, whereby the state of the user having difficulty in selecting a color chip can be more effectively avoided, and the time to acquire the visual characteristic can be shortened.

The method for detecting the state of the user having difficulty in selecting the color chips and the display method to display the measurement chips 105 described in Example 5 are examples, and the present invention is not limited to these methods.

### OTHER EXAMPLES

The present invention can also be carried out by supplying a program to implement one or more functions of the above mentioned embodiments to a system or apparatus via a network or storage medium, and one or more processors of the computer of the system or apparatus reading and executing the program. The present invention can also be carried out by a circuit (e.g. ASIC) that implements one or more functions.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-006963, filed on Jan. 16, 2015, and Japanese Patent Application No. 2015-009587, filed on Jan. 21, 2015, which are hereby incorporated by reference herein in their entirety.

What is claimed is:

1. An arithmetic processor comprising:
a selection unit configured for a user to select a color that satisfies a predetermined condition with respect to a reference color formed on a medium that reflects light, out of a plurality of different colors displayed on a display apparatus;
a determination unit configured to determine colors to be displayed on the display apparatus next time, based on the color selected by the user; and
a calculation unit configured to calculate a visual characteristic of the user, based on results of repeating the determination of colors by the determination unit and the selection by the user for a plurality of times, wherein
in a case where the user repeatedly selects a color out of a combination of two or more colors for a predetermined number of times or more, the calculation unit calculates the visual characteristic of the user, based on the colors included in the combination.

2. The arithmetic processor according to claim 1, wherein
the color that satisfies the predetermined condition is a color closest to the reference color.

3. The arithmetic processor according to claim 1, wherein
the calculation unit calculates the visual characteristic of the user, based on the color determined by averaging the colors included in the combination.

4. The arithmetic processor according to claim 1, wherein
the calculation unit calculates the visual characteristic of the user, based on the color determined by weighting and adding the colors included in the combination.

5. The arithmetic processor according to claim 4, wherein
the calculation unit assigns a greater weight to a color, which the user selected at a later timing, than to other colors.

6. The arithmetic processor according to claim 4, wherein
the calculation unit weights and adds each color included in the combination by assigning a weight according to the number of times selected by the user.

7. The arithmetic processor according to claim 3, wherein
the calculation unit calculates the visual characteristic of the user, based on results of repeating determination of the colors by the determination unit and the selection by the user until the color differences between the color determined by averaging the colors included in the combination and the colors included in the combination become a threshold or less.

8. The arithmetic processor according to claim 4, wherein
the calculation unit calculates the visual characteristic of the user, based on results of repeating determination of the colors by the determination unit and the selection by the user until the color differences between the color determined by weighting and adding the colors included in the combination and colors included in the combination become a threshold or less.

9. The arithmetic processor according to claim 1, wherein
the determination unit determines a color selected by the user and a color having a predetermined color difference from the color, as colors to be displayed on the display apparatuses next time.

10. The arithmetic processor according to claim 9, wherein in a case where the color selected by the user is the same
as the color selected by the user last time, the determination unit decreases the color difference, which is set between the selected color and the other color, for the colors to be displayed on the display apparatuses next time.

11. The arithmetic processor according to claim 1, wherein the reference colors are printed colors.

12. A control method for an arithmetic processor,
the method comprising:
a selection step in which a user selects a color that satisfies a predetermined condition with respect to a reference color formed on a medium that reflects light, out of a plurality of different colors displayed on a display apparatus;
a determination step in which colors to be displayed on the display apparatus next time are determined, based on the color selected by the user; and
a calculation step in which a visual characteristic of the user is calculated, based on results of repeating the determination of colors in the determination step and the selection of colors by the user for a plurality of times, wherein
in a case where the user repeatedly selects a color out of a combination of two or more colors for a fixed number of times or more, the visual characteristic of the user is calculated, based on the colors included in the combination in the calculation step.

13. An arithmetic processor comprising:
a selection unit configured for a user to select a color chip that satisfies a predetermined condition with respect to a reference color out of a plurality of different color chips displayed on a display apparatus;
a determination unit configured to determine the color chip selected by the user and one or a plurality of color chips having a color difference from the color chip, as color chips to be displayed on the display apparatus next time; and
a calculation unit configured to calculate a visual characteristic of the user, based on history information, which is information on the results of repeating the determination of the color chips by the determination unit and the selection of a color chip by the user for a plurality of times, wherein
in a case where determination is made based on the history information that the color chips selected by the user are not converged to one, the determination unit changes the color chips to be displayed on the display apparatus.

14. The arithmetic processor according to claim 13, wherein
the color chip that satisfies the predetermined condition is a color chip closest to the reference color.

15. The arithmetic processor according to claim 13, wherein
in a case where the user repeatedly selects a plurality of fixed color chips, the determination unit determines that the color chips selected by the user are not converged to one.

16. The arithmetic processor according to claim 15, wherein
the determination unit determines a color chip of a central color, which is determined based on the plurality of fixed color chips, and a color chip of one or a plurality of vicinity colors having a color difference from the color chip of the central color, as the color chips to be displayed on the display apparatus next time.

17. The arithmetic processor according to claim 16, wherein
the determination unit determines the central color by averaging the colors of the plurality of fixed color chips.

18. The arithmetic processor according to claim 16, wherein
the determination unit decreases the color difference between the central color and the vicinity color as the vicinity color is presumed to be closer to the visual characteristic of the user.

19. The arithmetic processor according to claim 16, wherein
in a case where the central color of the color chips displayed on the display apparatus last time is the same as the central color determined based on the color chip selected by the user this time, the determination unit decreases the color difference between the central color and the vicinity colors of the color chips, which are to be displayed on the apparatus next time, compared with the last time.

20. The arithmetic processor according to claim 13, wherein
the determination unit determines that the selection of color chips by the user is not converged to one in a case where it takes a predetermined time or longer for the user to select a color chip.

21. The arithmetic processor according to claim 15, wherein
the determination unit determines color chips to be displayed on the display apparatus next time by changing hue of the color chips displayed on the display apparatus last time.

22. The arithmetic processor according to claim 15, wherein
the determination unit determines color chips to be displayed on the display apparatus next time by changing the number or size of the color chips displayed on the display apparatus last time.

23. The arithmetic processor according to claim 13, wherein
the reference color is a color formed on a medium that reflects light.

24. The arithmetic processor according to claim 13, wherein
the reference colors are colors displayed on the display apparatus.

25. The arithmetic processor according to claim 24, wherein
the display apparatus includes a light emitting unit,
the light emitting unit includes a first region that emits light having a broadband emission spectrum, and a second region that emits light having a narrowband emission spectrum,
the reference color is displayed on a display region of the display apparatus corresponding to the first region of the light emitting unit, and
the color chips are displayed on a display region of the display apparatus corresponding to the second region of the light emitting unit.

26. The arithmetic processor according to claim 25, wherein
in the first region of the light emitting unit, light emission having the broadband emission spectrum and light emission having the narrowband emission spectrum can be switched over, and the light having the broadband emission spectrum is emitted in a case where the visual characteristic of the user is acquired, and the light having the narrowband emission spectrum is emitted under normal conditions.

27. The arithmetic processor according to claim 26, wherein
the first region of the light emitting unit has a red LED, a green LED, a blue LED and a white LED as light sources,
the second region of the light emitting unit has a red LED, a green LED and a blue LED as light sources, and from the first region of the light emitting unit, the red LED, the green LED, the blue LED and the white LED emit light in a case where the visual characteristic of the user is acquired, and the red LED, the green LED and the blue LED emit light under normal conditions.

28. The arithmetic processor according to claim 26, wherein
the first region of the light emitting unit has a red LED, a green LED, a blue LED and a CCFL as light sources,
the second region of the light emitting unit has a red LED, a green LED and a blue LED as light sources, and
from the first region of the light emitting unit, the CCFL emits light in a case where the visual characteristic of the user is acquired, and the red LED, the green LED and the blue LED emit light under normal conditions.

29. A control method for an arithmetic processor configured to acquire a visual characteristic of a user,
the method comprising:
a selection step in which a user selects a color chip that satisfies a predetermined condition with respect to a reference color, out of a plurality of different color chips displayed on a display apparatus;
a determination step in which a color chip selected by the user and one or a plurality of color chips having a color difference from the color chip are determined as color chips to be displayed on the display apparatus next time; and
a calculation step in which the visual characteristic of the user is calculated, based on history information, which is information on results of repeating the determination of the color chips in the determination step and the selection of the color chip by the user for a plurality of times, wherein
in a case where determination is made that color chips selected by the user are not converged to one, based on the history information, the color chips to be displayed on the display apparatus are changed in the determination step.
